(19) United States
(12) Patent Application Publication
(10) Pub. No.: US 2024/0001487 A1
(43) Pub. Date: Jan. 4, 2024

(54) MEDICAL TREATMENT APPARATUS AND TREATMENT PROBE THEREOF

(71) Applicant: Shanghai HROCT LITT Medical Technology Co., LTD., Shanghai (CN)

(72) Inventors: Xiaofei CUI, Shanghai (CN); Dongdong ZHANG, Shanghai (CN); Yuqi FU, Shanghai (CN); Muhammad Nasir GULZARI, Shanghai (CN); Weibang BAI, Shanghai (CN); Shihang CHEN, Shanghai (CN); Moyang CHEN, Shanghai (CN); Yinyan WANG, Shanghai (CN); Peng WANG, Shanghai (CN); Lei ZHU, Shanghai (CN); Shengzhong LI, Shanghai (CN)

(73) Assignee: Shanghai HROCT LITT Medical Technology Co., LTD., Shanghai (CN)

(21) Appl. No.: 17/980,444

(22) Filed: Nov. 3, 2022

Related U.S. Application Data
(63) Continuation of application No. PCT/CN2022/123856, filed on Oct. 8, 2022.

(30) Foreign Application Priority Data
Jul. 2, 2022 (CN) 202210776011.3
Aug. 2, 2022 (CN) 202210924425.6
Sep. 21, 2022 (CN) 202211151254.4
Sep. 22, 2022 (CN) 202211156542.9
Sep. 22, 2022 (CN) 202211159480.7

(51) Int. Cl. B23K 26/066; B23K 26/40

(52) U.S. Cl. CPC B23K 26/066; B23K 26/40; B23K 2103/50

(57) ABSTRACT

Disclosed is a medical treatment apparatus. The medical treatment apparatus includes a magnetic resonance imaging (MRI) device, configured for imaging of a specific region including a target object and generating a magnetic resonance image; a laser interstitial thermal therapy (LITT) device, including an LITT probe, the LITT probe being positioned close to the target object based on the magnetic resonance image, and being configured to treat the target object by emitting laser; and a temperature measurement element, the temperature measurement element and the LITT probe being integrated as an integrated probe, and the temperature measurement element being configured to obtain a temperature of the target object.

### CROSS-REFERENCE TO RELATED APPLICATIONS

This application is a continuation of International Application No. PCT/CN2022/123856, filed on Oct. 8, 2022, and claims priority to Chinese Patent Application No. 202210776011.3, filed on Jul. 2, 2022, Chinese Patent Application No. 202210924425.6, filed on Aug. 2, 2022, Chinese Patent Application No. 202211159480.7, filed on Sep. 22, 2022, Chinese Patent Application No. 202211156542.9, filed on Sep. 22, 2022, and Chinese Patent Application No. 202211151254.4, filed on Sep. 21, 2022, the entire contents of each of which are hereby incorporated by reference.

### TECHNICAL FIELD

This application relates to medical diagnosis and treatment, and more particularly, relates to a medical treatment apparatus and treatment probes thereof.

### BACKGROUND

Laser interstitial thermal therapy (LITT) is a treatment technology that uses thermal effect of laser to destroy target tissue, and is a latest minimally invasive surgery for brain tumors. The basic principle of LITT is to introduce an optical fiber probe integrating a cooling circulation tube into a lesion in the brain of a patient in a stereotactic manner in neurosurgery. Laser is transmitted to the probe through an optical fiber during a treatment, so as to heat lesion tissue around the probe, thereby realizing ablation of the lesion tissue. In the meanwhile, an LITT process is magnetic resonance guided (MRg) so as to determine an ablation target.

The present LITT system has the following problems: (1) magnetic resonance thermal imaging (MRTI) is used to measure temperature(s) of a far end of an LITT probe and tissue around the ablation target. A real time temperature control and an accuracy of examination can hardly be ensured due to a large distance to the target lesion. An additional algorithm estimation is usually needed for a nonreal time compensation, which results in a sluggish algorithm theory subjectivity, and brings about difficulties in quantifying and avoiding a heat damage on surrounding healthy tissue; (2) a high-power LITT diffusion applicator, used as a core structure of the LITT probe, lacks uniformity in the manufacturing process. Physical scattering units of the LITT diffusion applicator are overly centralized. Thus, vectorial optical energy generated by the LITT diffusion applicator can hardly irradiate a surrounding target to clot or even ablate pathological tissue thoroughly, resulting in frequent overtreatments in partial regions and incompleteness in other partial regions. The treatment is not fulfilled thoroughly, excessive regional carbonization may exist, and an acceptable curative effect can hardly be achieved; (3) the high-power LITT lacks a mechanism that evaluates the efficacy of the ablation accurately. During the ablation, magnetic resonance imaging (MRI) is solely relied on to perform millimeter-level macroscopical imaging for a rough edge identification. Tumor relapse or residue is evaluated in combination with return visits for MRI in the coming months after a patient is discharged, which results in an enormous risk on whether the patient is eligible to endure a re-operation in case of a relapse; (4) arrestment of the high-power LITT is mainly dependent on manual arrestment by a doctor, which has difficulties in controlling a retracement accuracy, performing a rotation operation, realizing a compatibility with lateral ablation for the irregular tumors, and treating highly irregular tumors, healthy nerve functional regions, or sensitive parts adjacent to the ablation position, thereby delaying a surgical treatment, and causing severe postoperative sequelae.

Thus, it is desirable to develop a new LITT device to solve the above problems, realize an integration of imaging diagnosis and ablation treatment effectively, and enhance the accuracy and a success rate of the treatment.

### SUMMARY

An aspect of the present disclosure provides a laser interstitial thermal therapy (LITT) lateral ablation probe, which is used in an LITT device. The LITT lateral ablation probe includes: a probe main body; and a coating layer covered on an end surface of the probe main body, wherein the end surface of the probe main body and an axis of the probe main body form a first angle, the probe main body includes a probe core and a hard cladding structure located at an outer peripheral of the probe core, the probe core is made of pure silicon oxide material, the hard cladding structure is made of technology enhanced clad silica (TECS) material, and the coating layer includes noble metal.

In some embodiments, the coating layer has a dual-layer structure, a layer of the dual-layer structure being close to the end surface of the probe main body includes pure silver, and the other layer of the dual-layer structure being away from the end surface of the probe main body includes silicon monoxide.

In some embodiments, the LITT lateral ablation probe is obtained by brightening and cleaning the end surface of the probe main body, and then further covering the coating layer on the end surface through magnetron sputtering.

Another aspect of the present disclosure provides a laser interstitial thermal therapy (LITT) lateral ablation probe, which is used in an LITT device. The LITT lateral ablation probe comprises a probe main body; and a lens connected to an end surface of the probe main body, wherein the end surface of the probe main body is perpendicular to an axis of the probe main body, the lens has a first vertex angle, the probe main body includes a probe core and a hard cladding structure located at an outer peripheral of the probe core, and the lens is a sapphire lens.

In some embodiments, the lens is a chamfered cylindrical lens, or a chamfered hemi-spherical or hemi-ellipsoidal lens.

In some embodiments, the LITT lateral ablation probe is obtained by brightening and cleaning the end surface of the probe main body, connecting the probe main body and the lens through fusion welding, and processing a welded surface between the probe main body and the lens using fire polishing.

A further aspect of the present disclosure provides a laser interstitial thermal therapy (LITT) circumferential ablation probe, which is used in an LITT device. The LITT circumferential ablation probe comprises a probe main body, the main body of the probe including a cone-shaped surface, one or more grooves being set on the cone-shaped surface, the one or more grooves being evenly distributed on the cone-shaped surface in an arabesquitic pattern, wherein the LITT circumferential ablation probe emits laser out of the one or more grooves.

In some embodiments, a diameter of the cone-shaped surface decreases from an initial diameter to a preset diameter, the initial diameter being 630 μm, and the preset diameter being 100 μm.

In some embodiments, the arabesquitic pattern includes a single thread pattern, a cross-thread shape, or a rhombic grid pattern, or a combination thereof.

In some embodiments, the cone-shaped surface is formed by performing operations including fixing two ends of a first workpiece with cone-shaped surface to be carved on a sliding rail and a fixing device, respectively; controlling, by a laser device controller, a laser device, to emit laser, wherein a power of the emitted laser is 30 W, a wavelength of the emitted laser is 10600 nm, the emitted laser is attenuated to 6.6-11.2 W by a laser power attenuator, the attenuated laser is split into two laser beams by a diffraction splitting lens unit, and the two laser beams have equal power and are focused onto a surface of the first workpiece by a focusing lens unit; and driving, by a motion driver, the first workpiece to move, the motion driver being controlled by a motion driver controller.

In some embodiments, the one or more grooves that are evenly distributed on the cone-shaped surface in the arabesquitic pattern are formed by performing operations including fixing two ends of a second workpiece with arabesquitic pattern to be carved on a sliding rail and a fixing device, respectively; controlling, by a laser device controller, a laser device, to emit laser, wherein a power of the emitted laser is 30 W, a wavelength of the emitted laser is 10600 nm, the emitted laser is focused on a surface of the second workpiece by a lens set, the lens set includes a first concave lens, a first convex lens, and a second concave lens, the first concave lens and the first convex lens are used to expand a diameter of the emitted laser into a first diameter, the second concave lens is used to focus the laser beam of the first diameter to form a spot on the surface of the second workpiece, and the spot has a diameter of 45 μm; and driving, by a motion driver, the second workpiece to move, the motion driver being controlled by a motion driver controller.

In some embodiments, a spatial intensity distribution of the laser emitted by the LITT circumferential ablation probe is obtained through operations including obtaining signals output by a laser measurement sensor, and generating a polar intensity of a specific orientation of the laser emitted by the LITT circumferential ablation probe, wherein the LITT circumferential ablation probe is connected to a helium-neon laser device that generates laser with a wavelength of 632.8 nm, a slit diaphragm is set between the laser measurement sensor and the LITT circumferential ablation probe, and a slit size of the slit diaphragm is 0.3 mm; and determining the spatial intensity distribution of the laser emitted by the LITT circumferential ablation probe by driving, by a motion driver, the slit diaphragm and the laser measurement sensor to move along a circumferential direction and an axial direction of the cone-shape surface of the LITT circumferential ablation probe, the motion driver being controlled by a motion driver controller, wherein the slit diaphragm and the laser measurement sensor are fixedly connected to the motion driver.

A further aspect of the present disclosure provides a medical treatment apparatus. The medical treatment apparatus may comprise a magnetic resonance imaging (MRI) device, configured for imaging of a specific region including a target object and generating a magnetic resonance image; a laser interstitial thermal therapy (LITT) device, including an LITT probe, the LITT probe being positioned close to the target object based on the magnetic resonance image, and configured to treat the target object by emitting laser; and a temperature measurement element, the temperature measurement element and the LITT probe being integrated as an integrated probe, and the temperature measurement element being configured to obtain a temperature of the target object, wherein the LITT probe includes an LITT lateral ablation probe or an LITT circumferential ablation probe.

In some embodiments, the LITT lateral ablation probe includes a probe main body; and a coating layer covered on an end surface of the probe main body, wherein the end surface of the probe main body and an axis of the probe main body form a first angle, the probe main body includes a probe core and a hard cladding structure located at an outer peripheral of the probe core, and the coating layer includes noble metal.

In some embodiments, the coating layer has a dual-layer structure, a layer of the dual-layer structure being close to the end surface of the probe main body includes pure silver, and the other layer of the dual-layer structure being away from the end surface of the probe main body includes silicon monoxide.

In some embodiments, the LITT lateral ablation probe is obtained by brightening and cleaning the end surface of the probe main body, and then further covering the coating layer on the end surface through magnetron sputtering.

In some embodiments, the LITT lateral ablation probe includes a probe main body; and a lens connected to an end surface of the probe main body, wherein the end surface of the probe main body is perpendicular to an axis of the probe main body, the lens has a first vertex angle, the probe main body includes a probe core and a hard cladding structure located at an outer peripheral of the probe core, and the lens is a sapphire lens.

In some embodiments, the lens is a chamfered cylindrical lens, or a chamfered hemi-spherical or hemi-ellipsoidal lens.

In some embodiments, the LITT lateral ablation probe is obtained by brightening and cleaning the end surface of the probe main body, connecting the probe main body and the lens through fusion welding, and processing a welded surface between the probe main body and the lens using fire polishing.

In some embodiments, the LITT circumferential ablation probe includes a probe main body, the main body of the probe including a cone-shaped surface, one or more grooves being set on the cone-shaped surface, the one or more grooves being evenly distributed on the cone-shaped surface in an arabesquitic pattern, wherein the LITT circumferential ablation probe emits laser out of the one or more grooves.

In some embodiments, a diameter of the cone-shaped surface decreases from an initial diameter to a preset diameter, the initial diameter being 630 μm, and the preset diameter being 100 μm.

In some embodiments, the arabesquitic pattern includes a single thread pattern, a cross-thread shape, or a rhombic grid pattern, or a combination thereof.

In some embodiments, the cone-shaped surface is formed by performing operations including fixing two ends of a first workpiece with cone-shaped surface to be carved on a sliding rail and a fixing device, respectively; controlling, by a laser device controller, a laser device, to emit laser, wherein a power of the emitted laser is 30 W, a wavelength of the emitted laser is 10600 nm, the emitted laser is attenuated to 6.6-11.2 W by a laser power attenuator, the attenuated laser is split into two laser beams by a diffraction splitting lens unit, and the two laser beams have equal power and are focused onto a surface of the first workpiece by a focusing lens unit; and driving, by a motion driver, the first workpiece to move, the motion driver being controlled by a motion driver controller.

In some embodiments, the one or more grooves that are evenly distributed on the cone-shaped surface in the arabesquitic pattern are formed by performing operations including fixing two ends of a second workpiece with arabesquitic pattern to be carved on a sliding rail and a fixing device, respectively; controlling, by a laser device controller, a laser device, to emit laser, wherein a power of the emitted laser is 30 W, a wavelength of the emitted laser is 10600 nm, the emitted laser is focused on a surface of the second workpiece by a lens set, the lens set includes a first concave lens, a first convex lens, and a second concave lens, the first concave lens and the first convex lens are used to expand a diameter of the emitted laser into a first diameter, the second concave lens is used to focus the laser beam of the first diameter to form a spot on the surface of the second workpiece, and the spot has a diameter of 45 μm; and driving, by a motion driver, the second workpiece to move, the motion driver being controlled by a motion driver controller.

In some embodiments, a spatial intensity distribution of the laser emitted by the LITT circumferential ablation probe is obtained through operations including obtaining signals output by a laser measurement sensor, and generating a polar intensity of a specific orientation of the laser emitted by the LITT circumferential ablation probe, wherein the LITT circumferential ablation probe is connected to a helium-neon laser device that generates laser with a wavelength of 632.8 nm, a slit diaphragm is set between the laser measurement sensor and the LITT circumferential ablation probe, and a slit size of the slit diaphragm is 0.3 mm; and determining the spatial intensity distribution of the laser emitted by the LITT circumferential ablation probe by driving, by a motion driver, the slit diaphragm and the laser measurement sensor to move along a circumferential direction and an axial direction of the cone-shape surface of the LITT circumferential ablation probe, the motion driver being controlled by a motion driver controller, wherein the slit diaphragm and the laser measurement sensor are fixedly connected to the motion driver.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings. In the drawings:

FIG. 1 illustrates a medical treatment system according to some embodiments of the present disclosure;

FIGS. 2A-2D illustrate exemplary structure diagrams of a medical treatment apparatus according to some embodiments of the present disclosure;

FIGS. 3A and 3B are structural diagrams of exemplary driving devices according to some embodiments of present disclosure;

FIGS. 4A-4C are schematic diagrams of an exemplary translation control mechanism and/or rotation control mechanism according to some embodiments of present disclosure;

FIG. 5 is an exemplary structural diagram of the control device according to some embodiments of present disclosure;

FIG. 6 illustrates two exemplary integrated probes of different types according to some embodiments of present disclosure;

FIG. 7 illustrates an exemplary LITT lateral ablation probe manufactured using a first preparation technology according to some embodiments of present disclosure;

FIG. 8 illustrates an exemplary LITT lateral ablation probe manufactured using a second preparation technology according to some embodiments of present disclosure;

FIG. 9 illustrates an exemplary LITT circumferential ablation probe according to some embodiments of present disclosure;

FIG. 10 is a schematic diagram illustrating the processing of a cone-shaped surface according to some embodiments of present disclosure;

FIG. 11 is a schematic diagram illustrating the processing of one or more grooves distributed in a cross-thread pattern according to some embodiments of present disclosure;

FIG. 12 is a schematic diagram illustrating a test of a distribution of vectorial optical energy of an LITT circumferential ablation probe according to some embodiments of the present disclosure;

FIG. 13 is a schematic diagram illustrating temperature measurement of a thermocouple according to some embodiments of the present disclosure;

FIG. 14 is a schematic diagram illustrating temperature measurement of an FBG sensor according to some embodiments of the present disclosure;

FIG. 15 illustrates an exemplary OCT probe according to some embodiments of the present disclosure;

FIG. 16 is a schematic diagram illustrating manufacturing of an FBG sensor according to some embodiments of the present disclosure;

FIG. 17 is a schematic diagram of a medical treatment apparatus according to some embodiments of the present disclosure;

FIG. 18 is a schematic diagram of an LITT photon thermometric probe according to some embodiments of the present disclosure;

FIG. 19 is a schematic diagram of an LITT photon ablation probe according to some embodiments of the present disclosure; and

FIG. 20 is a schematic diagram of an LITT photon ablation probe and an LITT photon thermometric probe during a treatment according to some embodiments of the present disclosure.

### DETAILED DESCRIPTION OF THE EMBODIMENTS

In order to explain the technical solutions of the embodiments of the present disclosure more clearly, the figures used in the descriptions of the embodiments will be introduced briefly. Obviously, the figures described in the following disclosure are merely examples or embodiments of the present specification, for those skilled in the art, the present specification may be applied to other scenarios according to the figures without creative efforts. Unless obviously stated in the context or expressively indicated otherwise, same numerals in the drawings represent same structures or operations.

It will be understood that the “system”, “apparatus”, “unit”, and/or “module” used in the present disclosure are one method to distinguish different components, elements, parts, section or assembly of different levels. However, the terms may be displaced by another expression if they may achieve the same purpose.

As described in the specification and claims, unless the context clearly indicates otherwise, the terminology “one”, “a”, “an” and/or “the” are not specially referred to the singular form, it may include plural forms as well. Generally speaking, the terms “include” and “comprise” specify the presence of clearly identified steps and elements, but the steps and elements do not constitute an exclusive listing, and the method or apparatus may also include other steps or elements.

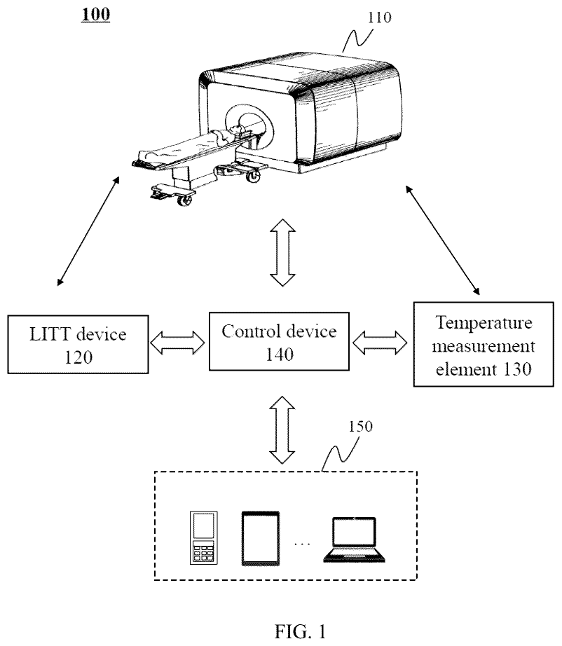

FIG. 1 illustrates a medical treatment system according to some embodiments of the present disclosure.

The medical treatment system 100 is used for medical diagnosis and/or treatment for a target object. The target object may include a biological subject (e.g., a human, an animal, etc.). For example, the target object may include a human body or specific parts thereof, such as the head, and/or tissue or lesions (such as brain tumors and epileptic foci) to be treated, or the like, or a combination thereof. In order to facilitate better understanding of the structure of the medical treatment system 100, a magnetic resonance guided (MRg) laser interstitial thermal therapy (LITT) is taken as an example in the following disclosure to describe the medical therapy system 100. The MRg-LITT may determine a position and a size of the target object (e.g., a tumor) through magnetic resonance imaging (MRI). Emission of laser may be guided to apply a thermal therapy to the target object effectively and controllably based on the position and size of the target object. However, the medical therapy system 100 is not limited to the MRG-LITT, the medical therapy system 100 may also be a treatment system of other types, such as an MRg-microwave therapy, etc.

Merely for illustration, as shown in FIG. 1, the medical treatment system 100 includes an MRI device 110, an LITT device 120, a temperature measurement element 130, a control device 140, and a terminal 150.

The MRI device 110 may be configured to scan a specific region including the target object and generate one or more MRI images. MRI device 110 may include an MRI scanner. The MRI scanner includes a magnet module and a radiofrequency (RF) module. The magnet module may include a main magnetic field generator and/or a gradient magnetic field generator. The main magnetic field generator may include a main magnet that generates a static magnetic field B0 during an MRI scanning process. The main magnet may use a permanent magnet, a superconducting electromagnet, a resistance electromagnet, etc. The gradient magnetic field generator may generate magnetic field gradients in the X direction, Y direction, and/or Z direction. The X direction used herein may also be referred to as a readout (RO) direction. The Y direction used herein may also be referred to as a phase encoding (PE) direction. The Z direction used herein may also be referred to as a slice selection (SS) direction. The gradient magnetic field may encode spatial information of the target object. The RF module may include one or more RF transmitting coils and/or one or more receiving coils. The one or more RF transmitting coil may emit RF signals (also referred to as radio frequency pulses) to stimulate a region of interest, and generate MRI signals. The one or more RF receiving coils may receive echo signals emitted from the region of interest. In some embodiments, the RF signals may be 30° pulse signals, 40° pulse signals, 60° pulse signals, etc. The MRI device 110 may process the MRI signals, and generate the one or more MRI images. The one or more MRI images may represent anatomical structure information of the specific region including the target object.

In some embodiments, the function, size, type, geometric shape, location, and quantity of each of the magnet module and/or the RF module may be determined or changed according to one or more specific conditions. For example, according to differences in view of the function and size, RF coils may be classified into body coils and local coils. The body coils may be set to birdcage coils, horizontal electromagnetic coils, saddle coils, etc. The local coil may include phased array coils, loop coils, etc. In some embodiments, the local coil may include a Helmholtz head coil. The Helmholtz head coil may create a uniform magnetic field of a small-scale surrounding the patient's head, and transmit RF signals and receive corresponding MRI signals of the head. The MRI signals of the head may be used for imaging of the patient's head and generating one or more MRI images of the head.

In some embodiments, the MRI device 110 may also obtain thermal image data for magnetic resonance thermal imaging (MRTI) from the MRI scanner. Based on the thermal image data, one or more thermal images may be reconstructed. The thermal images may represent temperature variations in the specific region including the target object. In some embodiments, each pixel/voxel in the thermal image may measure temperature variations at a corresponding position in the specific region.

The LITT device 120 may be configured to emit laser, and treat the target object, e.g., ablate the target object, through thermal effect of the laser. A position and size of the target object (e.g., tumor) may be determined based on the above MRI images and/or thermal images. Therefore, the LITT device 120 may be guided to emit laser effectively and controllably to apply the thermal therapy to the target object.

The LITT device 120 includes a laser device, an LITT probe, and one or more channels (such as fibers) and interfaces that connect the laser device and the LITT probe. The laser device may be used to generate laser. The laser device may be a solid laser device, a gas laser device, a liquid laser device, a semiconductor laser device, a free electron laser device, etc. In some embodiments, the wavelength of the laser generated by the laser device may be within a certain range, such as near infrared region (0.75-2.5 micrometers (μm)). The laser emitted by the laser device transmits to the LITT probe through the one or more fibers and interfaces.

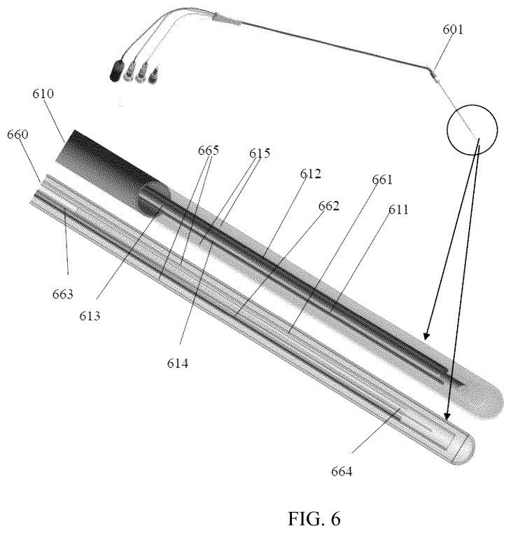

The LITT probe, also referred to as LITT diffusion applicator, is used as a treatment probe to deliver the laser generated by the laser device to the target object to achieve ablation of a lesion. The LITT probe includes a probe main body. The probe main body may be set as different structures. For different structures, position(s) where the laser emits out of the LITT probe may be different. Specifically, depending on the position(s) where the laser emits out of the LITT probe, the LITT probe may be divided into an LITT lateral ablation probe and an LITT circumferential ablation probe. The LITT lateral ablation probe refers to an LITT probe that the position where the laser emits out of the LITT probe is at an end (far end) of the LITT probe. At this time, the laser emits out of the LITT probe in a direction the same as or at a certain angle with an axis of the LITT probe. The LITT circumferential ablation probe refers to an LITT probe that the positions where the laser emits out of the LITT probe are evenly distributed on a periphery of the LITT probe. At this time, the laser emits out of the LITT probe in radial directions of the LITT probe that are spreading uniformly around the LITT probe. Details regarding the types, structures, and manufacturing technologies of the LITT probes may be referred to other figures (e.g., FIGS. 6-11) and the descriptions thereof, which are not repeated here.

The temperature measurement element 130 may be used to measure temperature(s) of the target object or tissue around the target object and/or the LITT probe. For example, the temperature measurement element 130 may be positioned close to the target object, and obtain temperatures of the target object and tissue around the target object. Based on the temperatures of the target object and the tissue around the target object, the LITT device 120 may deliver heat to the target object through the LITT probe in a controlled manner, treat the target object, and protect healthy tissue around the target object.

The temperature measurement element 130 may include a thermocouple, a fiber Bragg grating (FBG) sensor. The thermocouple may measure the temperature directly. The thermocouple may convert a temperature signal into a thermal electromotance signal. The thermal electromotance signal may be further converted into the temperature of the measured object. The thermocouple may include a K-type thermocouple, a T-type thermocouple, an E-type thermocouple, an S-type thermocouple, a B-type thermocouple, etc. In some embodiments, the thermocouple is the K-type thermocouple.

The control device 140 may control one or more components of the medical treatment system 100 so as to perform corresponding operations. The control device 140 may generate corresponding instructions based on controlled components and operations to be performed. The instructions are transmitted to the controlled components in the form of electric signals, so that the controlled components may perform the corresponding operations. For example, the control device 140 may receive request or command information input through the terminal 150, and information (such as image, temperature data, etc.) generated from the MRI device 110 and/or the temperature measurement element 130. The control device 140 may generate control instructions based on the above information. The control instructions may be sent to the LITT device 120 for treatment of the target object.

In some embodiments, the control device 140 may be a microcontroller (MCU), a central processor (CPU), a programmable logic device (PLD), an application specific integrated circuit (ASIC), a single-chip microcomputer (SCM), a system on chip (SOC), etc.

The terminal 150 may be used for input/output of information (e.g., image, data, etc.). The terminal 150 may include a computer, a mobile device (such as a mobile phone, a tablet, a laptop), etc., or any combination thereof. In some embodiments, the terminal 150 may include a wearable equipment, a virtual reality equipment, an augmented reality equipment, or any combination thereof. The wearable device may include a bracelet, a glasses, a helmet, a watch, or the like, or any combination thereof. The virtual reality device and/or augmented reality device may include a virtual reality helmet, a virtual reality glasses, a virtual reality eye mask, an augmented reality helmet, an augmented reality glasses, an augmented reality eye mask, or the like, or any combination thereof. For example, the virtual reality device and/or augmented reality device may include Google Glass™, Oculus Rift™, HoloLens™, Gear VR™, etc. In some embodiments, the terminal 150 may be part of the control device 140.

The devices or components of the medical treatment system 100 may be local devices or components, or remote devices or components. The devices or components may be connected in one or more ways. Merely by way of example, the control device 140 may be a remote device. The LITT device 110 and the MRI device 120 may be connected to the control device 140 through a network. As another example, the control device 140 may be a local device. The LITT device 110 and the MRI device 120 may be connected to the control device 140 directly. As a further example, the terminal 150 may be connected to control device 140 directly or through the network. The network may include any appropriate network that facilitates information and/or data exchange of the medical treatment system 100. In some embodiments, the network may be and/or include a public network (such as Internet), a private network (such as a local regional network (LAN), a wide area network (WAN)), a wired network (such as Ethernet), a wireless network (such as 802.11 network, Wi-Fi network), a cellular network (such as long-term evolution (LTE) network), a frame relay network, a virtual private network (VPN), a satellite network, a telephone network, a router, a hub, an exchanger, a server computer and/or any combination thereof. Merely by way of example, the network may include a cable network, a wired network, a fiber network, a telecommunication network, an internal network, a wireless local area network (WLAN), a metropolitan area network (MAN), a public switched telephone network (PSTN), a Bluetooth™ network, a Zigbee™ network, a near-field communication (NFC) network, or any combination thereof.

It should be noted that the descriptions of the medical treatment system 100 and the components/modules are merely for the convenience of description, and not intended to limit the application within the scope of the implementation of the embodiments. It is understood that for person of ordinary skill in the art, after understanding the principle of the system, various modifications and changes on the medical treatment system 100 may be made without departing from this principle. For example, a combination of any modules, or a connection of a subsystem to other modules may also be implemented. However, these modifications and changes are still within the scope of this disclosure.

For example, the medical treatment system 100 may also include an optical coherence tomography (OCT) device (not shown in the figure). The OCT device may use low-coherence interference of a broad band light source for imaging of the target object, and generate an OCT image of a high-resolution (e.g., micron level) and/or an ultra-large depth. The OCT device includes a time domain optical coherence tomography (TDOCT) device, spectrum domain optical coherence tomography (SDOCT) device and/or a swept source optical coherence tomography (SSOCT) device.

In some embodiments, the OCT device includes a light source, an OCT probe, an interference component, and optical fibers and interfaces that connect the various components. The light source uses a low-coherence light source to improve a vertical resolution of the OCT imaging. The OCT probe is used to emit light generated by the light source to the target object and receive reflected light. The low-coherence light generated by the light source may include reference light and sample light (emitted from the OCT probe). The reference light and sample light reflected (or back reflected) by a reference mirror and tissue of the target object, respectively, may interfere via the interference component. The OCT device (e.g., an electro-optical system thereof) may obtain depth information of structure of the target object based on an interference spectrum generated by in the interference. The OCT image of the target object may be generated based on the depth information. The OCT image may be a two-dimensional or three-dimensional image. The OCT device may provide pathological imaging. In combination with the LITT device, the OCT device may detect tumor residue of the ablated lesion in real time, and implement imaging of the cancerous pathology remained at an edge of the ablated lesion, such that a fast supplementary ablation may be performed, and the tumor residue may be reduced and a recurrence probability may be minimized.

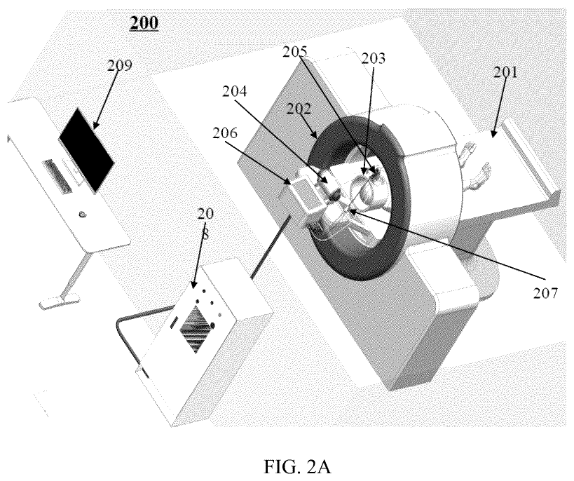

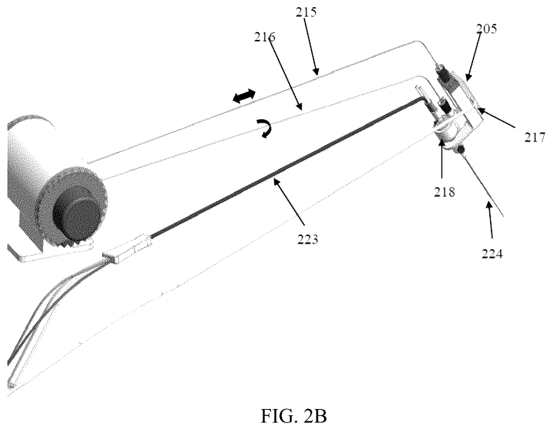

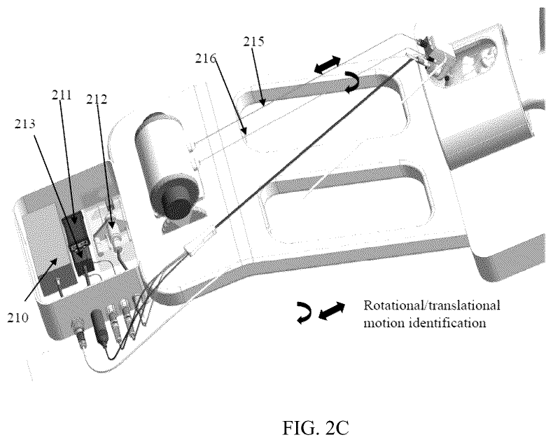

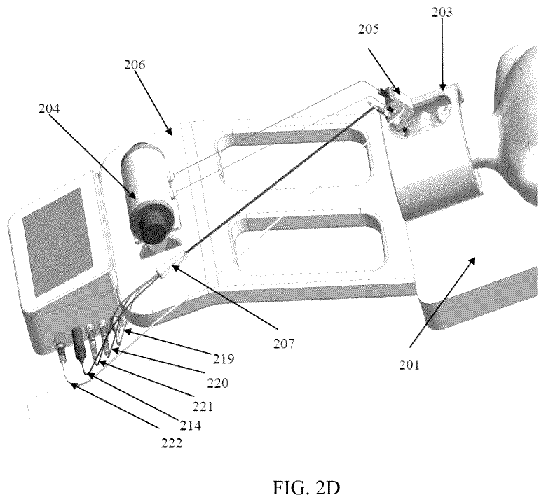

FIGS. 2A-2D illustrate exemplary structural diagrams of a medical treatment apparatus according to some embodiments of the present disclosure.

In some embodiments, the medical treatment apparatus 200 may be an apparatus that integrates the various devices and components (local or remote) of the medical treatment system 100. As illustrated in the figures, the medical treatment apparatus 200 may include a supporting and fixing platform 201, an MRI device 202, a head coil 203, an LITT device (not shown in the figure), and an OCT device (not shown in the figure), a temperature measurement element (not shown in the figure), a cooling device (not shown in the figure), a driving device 204, an interface platform 205, a control relay platform 206, an integrating element 207, a control device 208, and a terminal 209.

The supporting and fixing platform 201, also referred to as a treatment bed, may support and/or fix a patient, and prevent a movement of the patient or a specific part thereof during a treatment. In some embodiments, the supporting and fixing platform 201 may move (e.g., translate, tilt, rotate, etc.) during a medical diagnosis and/or treatment according to control instructions issued by the control device 208, so as to adjust a position and posture of the patient. For example, the supporting and fixing platform 201 may translate or tilt based on control instructions issued by the control device 208 in an MRI scanning process, so as to provide a better baseline of scanning positions in MRI.

The MRI device 202 may generate one or more MRI images by imaging a specific region including the target object (e.g., tumors in the patient's brain). The one or more MRI image may be real time MRI images or nonreal-time MRI images. The one or more MRI images may include a three-dimensional image or multiple two-dimensional images (e.g., a transverse image, a coronal image, and a sagittal image), and characterize information (such as a position, a size, etc.) of the target object in the three-dimensional space. The information of the target object in the three-dimensional space provided by the one or more MRI images may be used for planning, before a treatment for the patient, a route along which the LITT probe, the OCT probe, and/or the temperature measurement element arrive a position where the target object locates through body tissue of the patient (also referred to as probe planning). The information of the target object in the three-dimensional space provided by the one or more MRI images may also be used for guiding the LITT probe, OCT probe, and/or temperature measurement element to access the body tissue of the patient according to the planned route, and arrive the position where the target object locates (referred to as probe guidance). In some embodiments, the one or more MRI images may be displayed on the terminal 209. The probe planning and the probe guidance may be implemented using the terminal 209 (e.g., physical elements such as a touch screen, a mouse, a keyboard, etc., of the terminal 209) by a user, such as a doctor based on the one or more MRI images. In some embodiments, the probe planning and the probe guidance may also be implemented by the system automatically based on the one or more MRI images.

In some embodiments, the MRI device 202 may also perform MRTI of the specific region including the target object, and generate one or more thermal images. The one or more thermal images may be registered with the one or more MRI images. Anatomical structure information of the specific region including the target object and temperature variation information of corresponding positions may be characterized by the registered thermal images and MRI images.

The head coil 203 may provide an MRI signal of the head of the patient in a relatively large depth. In some embodiments, the head coil 203 is Helmholtz magnetic coil. When the target object (for example, a tumor) is in the brain of the patient, the head coil 203 may emit RF signals and receive MRI signals about the head of the patient. The MRI signals received by the head coil 203 may have a higher signal-to-noise ratio (SNR) compared to the MRI signals received by the MRI device 202. Therefore, information of the target object provided by head MRI images reconstructed based on the MRI signals of the head received by the head coil 203 may be more accurate in comparison with that provided by the one or more MRI images. In this application, the head coil 203 may be coupled to the MRI device 202 through an interface so as to generate one or more head MRI images of high accuracies.

In some embodiments, the head coil 203 may also obtain thermal image data of the head for head MRTI. Based on the thermal image data of the head, one or more head thermal images of the patient may be reconstructed. In some embodiments, the head coil 203 may also support the head of the patient.

The LITT device may emit laser and treat the target object using thermal effect of the laser. The LITT device includes a laser device, an LITT probes (e.g., an LITT lateral ablation probe, an LITT circumferential ablation probe), and channels (such as fibers) and interfaces that connect the laser device and the LITT probe. In some embodiments, the laser device may include a tunable laser diode and/or an untunable laser diode. A power of the tunable laser diode is adjustable within a specific range, such as 0-500 W, 10-250 W, 50-150 W, etc. A power of the untunable laser diode is a specific value, for example, 10 W, 12 W, 15 W, 20 W, 30 W, 60 W, 100 W, 150 W, etc.

In some embodiments, the LITT device includes a LITT probe motion controller 210, which controls the movement of the LITT probe. During a treatment of the target object, the LITT probe motion controller 210 controls the LITT probe to pass through a probe channel configured on the interface platform 206, traverse the skull of the patient, access the brain of the patient, and arrive at a position where the target object locates according to the probe planning and probe guidance. The interface platform 206 may be fixed to the head of the patient. In some embodiments, the medical treatment apparatus 200 may include a motion sensor (not shown in the figure). The motion sensor provides real-time feedback of motion data of the LITT probe when the LITT probe moves. The motion sensor may be, for example, a piezoelectric sensor, an inductive sensor, an eddy-current sensor, etc. The motion sensor may be connected to the LITT probe. The motion sensor may be configured on the interface platform 206.

In some embodiments, the laser generated by the laser device is transmitted to the LITT probe through an optical relaying and processing device 211. The optical relaying and processing device 211 may adjust parameters (e.g., a power, a frequency, etc.) of the laser. For example, the optical relaying and processing device 211 may compensate an attenuation of the laser such that the power of the laser reaches a specific power, or perform an attenuation processing on the laser such that the power of the laser is reduced to meet the requirement of the medical treatment. The optical relaying and processing device 211 is configured on the control relay platform 206.

The OCT device may detect reflection signals, scattering signals, etc., of biological tissue based on light transmittance of biological structures, and convert the detected signals into electric signals to generate one or more OCT images. The one or more OCT images may be real time OCT images, or non-real time OCT images. The OCT device includes a light source, an OCT probe, an interference component, and optical fibers and interfaces that connect the various components of the OCT device. The light source uses a low-coherence light source to improve a vertical resolution of the OCT imaging. In this embodiment, the OCT device may be dual-mode OCT, and the light source may generate two light signals of different parameters (e.g., a bandwidth and a central wavelength). Merely by way of example, the dual-mode OCT generates a first light signal of a bandwidth exceeding 160 nm and a central wavelength at 840 nm, and a second light signal of a swept-frequency range exceeding 100 nm and a central wavelength at 1300 nm. The dual-mode OCT may facilitate pathological imaging with approximating 1 μm resolution and centimeter-level depth.

In a transmission path (for example, at a rotating joint) of the light signals generated by the OCT device, a fiber optic slip ring 212 may be used for the continuous transmission of the light signals. For the dual-mode OCT, a multi-channel fiber optic slip ring (such as a dual-channel fiber optic slip ring that adapts the two different central wavelengths of the light signals), also referred to as multi-mode fiber optic slip ring, may be used. The fiber optic slip ring 212 may be configured on the control relay platform 206.

The temperature measurement element may measure temperature(s) of the target object or tissue around the target object and/or the LITT probe. The temperature measurement element may include a thermocouple (such as K-type thermocouple) and an FBG sensor.

The FBG sensor is made of a specific material. The specific material for manufacturing the FBG sensor needs to satisfy the following parameter conditions. Merely for illustration purposes, the parameter conditions may include: a cut-off wavelength of the specific material ≤1280 nm, a maximum attenuation at 1310 nm≤0.35 dB/km, the maximum attenuation at 1625 nm≤0.23 dB/km, a fiber mode field diameter (MFD) at 1310 nm=9.2±0.4 μm, the fiber MFD at 1550 nm=10.4±0.5 μm, a chromatic dispersion at 1550 nm 18 ps/(nm·km), the chromatic dispersion at 1625 nm≤22 ps/(nm·km), a polarization mode dispersion LDV≤0.06, a maximum separate fiber 0.1, a point discontinuity at both 1310 nm and 1550 nm≤0.05 dB, an outer diameter of a cladding structure=125±0.3 μm, a concentricity between a fiber core and the cladding structure ≤0.3 μm, out-of-round of the cladding structure ≤0.7%, a fiber core diameter=8.2 μm, an outer diameter of an acrylic ester coating layer of the cladding structure=242±5 μm, a concentricity between the acrylic ester coating layer and the cladding structure <12 μm, coloring CD=250+15/−9 μm, fiber curl ≥5 curvature diameter m, a numerical aperture equals 0.12, a refractivity difference equals 0.36%, an effective group refractive index at 1310 nm equals 1.467, the effective refractive index at 1550 nm equals 1.4677, an anti-fatigue parameter=20, a Rayleigh backscattering coefficient at 1310 nm equals −77 dB, and the Rayleigh backscattering coefficient at 1550 nm equals −82 dB. In the meanwhile, the specific material also needs to satisfy a specific chromatic dispersion formula:

$$D ( λ ) \approx \frac{{S}_{0}}{4} [ λ - \frac{{λ}_{0}^{4}}{{λ}^{3}} ] ps / ( nm \cdot km ) ,$$

where 1200 nm≤λ≤1625 nm, the zero dispersion slope S₀≤0.088 ps/(nm·km), and the zero dispersion wavelength λ₀ is in a range 1304 nm≤λ₀≤1324 nm.

The specific material mentioned above may be exposed to ultraviolet light in a certain wavelength range (e.g., 240-244 nm, 244-248 nm, 248-252 nm, 252-256 nm, etc.), so that the refractive index of the fiber core is modulated periodically. The periodic core index modulations generate core modes. These core modes reflect or transmit through index boundaries and interfere with each other. In turn, input light is strongly reflected merely at a specific wavelength determined by a certain phase matching condition. The specific wavelength is referred to as a Bragg wavelength of FBG. The phase matching condition is also referred to as a Bragg condition. The FBG sensor is formed accordingly. The FBG sensor may be used for monitoring temperatures of interstitial tissue (e.g., the target object and tissue around the target object) and (a remote end of) the LITT probe in real time during a dispersive irradiation of laser ablation.

The cooling device may control the temperature of the LITT probe. Since the LITT probe emits laser and treats the target object using the thermal effect of the laser, the temperature of the LITT probe may increase during the treatment process with an extension of a treatment time. An excessive temperature will not only affect normal working of the LITT probe, but also damage normal tissue of the patient, which may cause postoperative sequelae. When the temperature of the LITT probe is too high, the cooling device transmits a specific cooling medium (for example, CO₂ gas) to the remote end of the LITT probe through a cooling channel 214 so as to cool down the LITT probe. The cooling device includes a cooling source (not shown), a cooling control element 213, and, a cooling channel 214. The cooling source stores the cooling medium. Merely by way of example, the cooling medium may be CO₂ gas. The cooling source may be a CO₂ gas holder for storing the CO₂ gas. The cooling control element 213 may control the opening and closing of the cooling source, parameters of the cooling medium (e.g., a flow rate and a pressure of the CO₂ gas), etc.

In some embodiments, the LITT probe, the OCT probe, the temperature measurement element and/or the cooling channel may be integrated as an integrated probe 224 that integrating medical diagnosis and treatment (also referred to as integrated probe 224 for brevity). For example, the temperature measurement element and the LITT probe may be integrated as the integrated probe 224. As another example, the OCT probe may be integrated into the integrated probe 224 together with the LITT probe and the temperature measurement element. As a further example, the LITT probe, the OCT probe, the temperature measurement element and the cooling channel may be integrated as the integrated probe 224.

The driving device 204 may drive the LITT probe (e.g., the integrated probe 224) to move to arrive at a position where the target object locates (probe accessing) or get away from the position where the target object locates (probe retracement). The motion of the LITT probe may include a translational motion and a rotational motion. In some embodiments, the driving device 204 includes a driving motor, one or more cables, and one or more motion control mechanisms. The LITT probe is connected to the motion control mechanisms. Through the one or more cables and the one or more motion control mechanisms, a force output by the driving motor may be conveyed to the integrated probe 224 to drive the integrated probe 224 to move. In this embodiment, as shown in FIG. 2B, the one or more cables include a translation cable 215 and a rotation cable 216. Correspondingly, the one or more motion control mechanisms include a translation control mechanism 217 and a rotation control mechanism 218. The translation control mechanism 217 is used to control the translational motion of the integrated probe 224. The rotation control mechanism 218 is used to control the rotational motion of the integrated probe 224. The translation cable 215 and the rotation cable 216 are connected with the translation control mechanism 217 and the rotation control mechanism 218, respectively. In some embodiments, the one or more cables (such as the translation cable 215 and the rotation cable 216) are specially made wires. The specially made wire has a relatively high stiffness and a relatively low elastic modulus, and is capable of transmitting a torque in a transmitting ratio of 1:1 in real time, thereby ensuring a motion accuracy of the LITT probe.

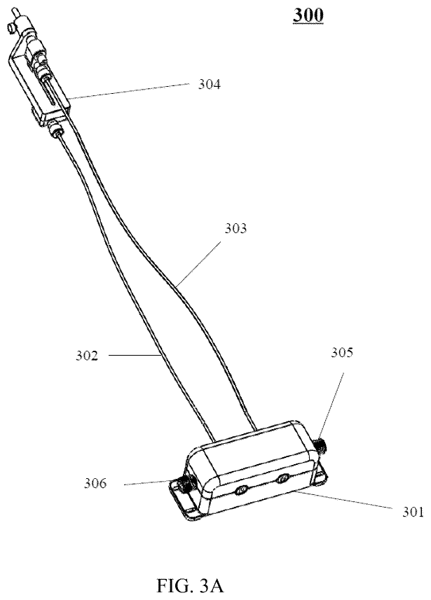

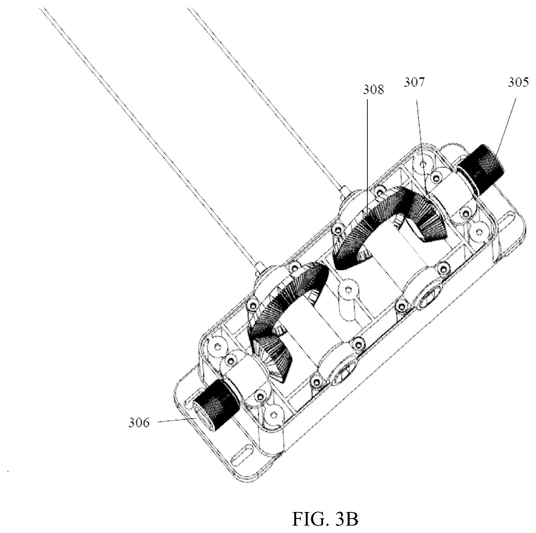

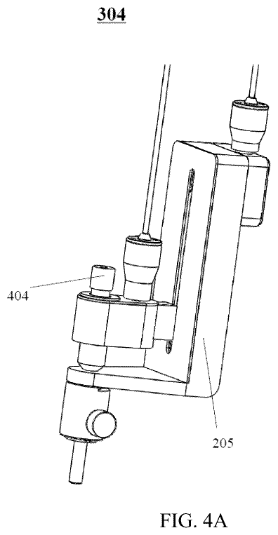

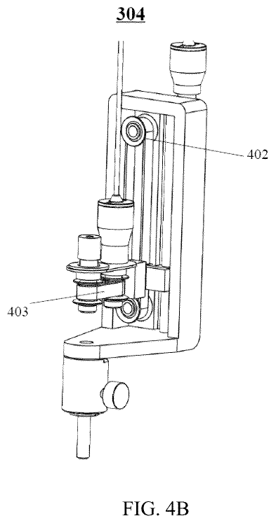

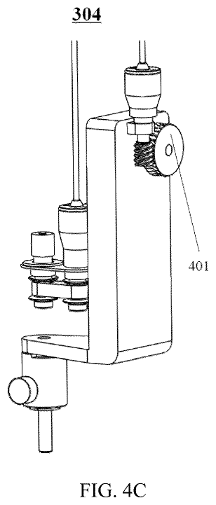

The driving motor may drive, according to actual needs, the motion of the translation cable 215 and/or the rotation cable 216. The motion of the translation cable 215 and/or the rotation cable 216 may control the translational motion and/or rotational motion of the integrated probe 224 through the translation control mechanism 217 and/or the rotation control mechanism 218. In this way, a precise motion control of the LITT probe (or the integrated probe 224) in two degrees of freedom may be realized. In some embodiments, the translation control mechanism 217 and the rotation control mechanism 218 may be configured on the interface platform 205. Details regarding specific structures and working principles of the driving device 204 may be refer to other figures (e.g., FIGS. 3 and 4) and the descriptions thereof in this application, which are not repeated here.

The interface platform 205 may carry one or more components or elements of the medical treatment apparatus 200. Merely for illustration, the components or elements carried on the interface platform 205 may include a probe insertion channel of the LITT probe, the motion sensor, and the one or more motion control mechanisms (such as the translation control mechanism 217 and the rotation control mechanism 218). In some embodiments, the interface platform 205 may be a dual-axis three-dimensional framework as shown in the figure. The interface platform 205 may be fixed to the head (skull) of the patient, and form a stable connection with the head of the patient to avoid a relative displacement between the interface platform 205 and the head of the patient. The components or elements carried on the interface platform 205 may be fixed to the dual-axis three-dimensional framework. The LITT probe (the integrated probe 224) may access the brain of the patient through the probe insertion channel provided on the interface platform 205 and arrive the position where the target object is located accurately.

The control relay platform 206 integrates a mid-section control or a relay control of each of one or more components or element of the medical treatment apparatus 200. For example, the control relay platform 206 integrates the LITT probe motion controller 210, the optical relaying and processing device 211, the OCT fiber optic slip ring 212, the cooling control element 213, and the driving device 204, such as driving motor.

As shown in FIG. 2C, a packaging box is configured on the control relay platform 206. The LITT probe motion controller 210, the optical relaying and processing device 211, the OCT fiber optic slip ring 212, and the cooling control element 213 are set in the packaging box.

As shown in the figure, five channels are led out of the packaging box, which includes an OCT probe channel 219 (optical fiber), an LITT probe channel 220 (optical fiber), a cooling channel 214, a control cable channel 221 of the temperature measurement element, and a cable channel 222 of the motion sensor. The OCT fiber optic slip ring 212 is connected to the OCT probe channel 219; the optical relaying and processing device 211 is connected to the LITT probe channel 220 and the control cable channel 221 of the temperature measurement element; the cooling control element 213 is connected to the cooling channel 214; and the LITT probe motion controller 210 is connected to the cable channel 222 of the motion sensor.

The integrating element 207 may integrate one or more signal control cables, optical channels, cooling channels, etc., of the medical treatment apparatus 200. In some embodiments, as shown in FIG. 2D, the integrating element 207 may integrate the OCT probe channel 219, the LITT probe channel 220, the cooling channel 214, the control cable channel 221 of the temperature measurement element, etc., in a manner of, for example, mechanical coupling. The integrating element 207 includes an integrating channel 223, which may accommodate the OCT probe channel 219, the LITT probe channel 220, the cooling channel 214, and the control cable channel 221 of the temperature measurement element. The integrating channel 223 may connect the integrated probe 224, for example, by connecting a shell of the integrated probe 224 in a sealed connection. By integrating the one or more signal control cables, optical channels, cooling channels, etc., of the medical treatment apparatus 200 via the integrating element 207, the transmission efficiency of the channels may be optimized, and an operation stability of the entire medical treatment apparatus 200 may be improved.

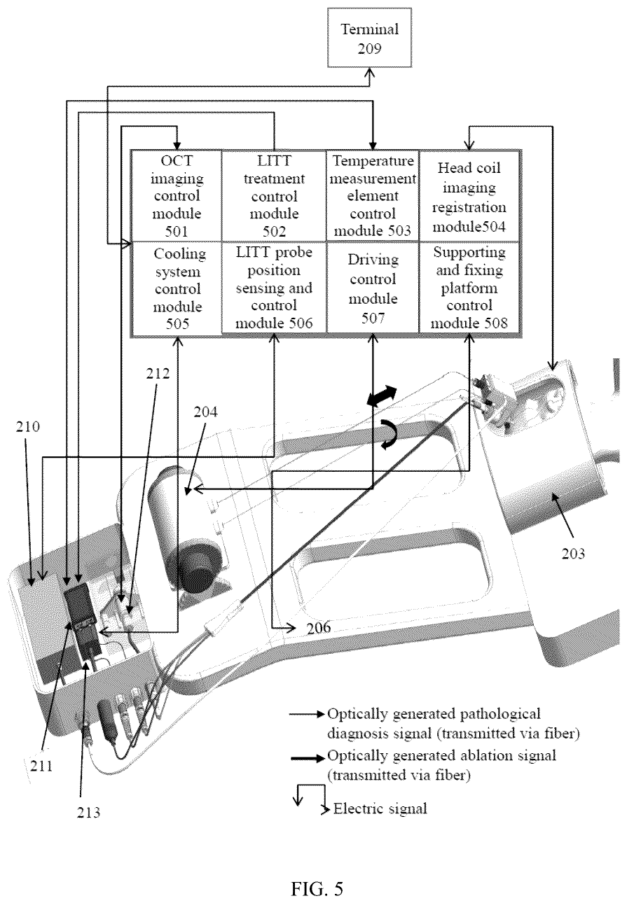

The control device 208 may control one or more components of the medical treatment apparatus 200 so as to perform corresponding operations. The control device 208 may generate corresponding instructions based on the one or more controlled components and operations to be performed. The instructions are transmitted to the one or more corresponding components in the form of electric signals, and prompt the one or more components to execute corresponding operations. In some embodiments, the control device 208 integrates an OCT imaging control module, an LITT treatment control module, a temperature measurement element control module, a head coil imaging registration module, a cooling system control module, an LITT probe position sensing and control module, a driving control module, a supporting and fixing platform control module, etc. Detailed modules and functions regarding the control device 208 may be referred to other figures (for example, FIG. 5) and the descriptions thereof, which are not repeated here.

The terminal 209 may display information regarding the various components of the medical treatment apparatus 200 and the patient in the form of images, data, etc. Merely for illustration, the terminal 209 may display one or more MRI images, one or more thermal images, one or more registration images of MRI images and thermal images of the target object, one or more OCT images, temperature information obtained by the temperature measurement element, and cooling information regarding the cooling device and the LITT probe. In some embodiments, the terminal 209 may also receive information input by a user. Information input through the terminal 209 may include an image, a number, text, voice, etc. For example, a user may input one or more operation instructions through the terminal 209. The operation instructions may include instructions for adjusting a position and a posture of the patient, instructions for setting working modes/parameters of the MRI device 202, the head coil 203, the LITT device (such as the LITT probe), the OCT device (such as the OCT probe), the cooling device, etc., instructions for probe planning during a treatment, etc. The information input through the terminal 209 may be sent to the control device 208. The control device 208 may generate control instructions for controlling corresponding devices or components to perform corresponding operations. In some embodiments, the terminal 209 may be or includes, for example, a computer, a mobile phone, a tablets computer, a console, etc.

All the components set forth above are compatible with MRI, which refers to application capabilities of the components in an MRI environment. Under a certain magnetic flux density, components that are compatible with MRI may not cause a significant interference to the MRI. Merely by way of example, as for a specific environment (for example, 0.5 T, 0.75 T, 1 T, 1.5 T, 2.0 T, 3.0 T, etc.), the operations of the components set forth above have no danger.

FIGS. 3A and 3B are structural diagrams of exemplary driving devices according to some embodiments of present disclosure.

As shown in FIG. 3A, a driving device 300 includes a driving force input end 301, cables 302 and 303, and a driving force output end 304. The driving force output end 304 is connected to the LITT probe (or the integrated probe 224). A driving force input from the driving force input end 301 may be delivered to the drive the driving force output end 304, and drive the LITT probe (or the integrated probe 224) to move. The driving force input end 301 may include a driving motor. The cables 302 and 303 includes a translation cable and a rotation cable, respectively. The cables 302 and 303 control the translational motion and rotational motion of the LITT probe (or the integrated probe 224), respectively.

Unlike FIGS. 2A-2D, the driving device 300 further includes driving knobs 305 and 306. As shown in FIG. 3B, the driving knob 305 is connected to the cable 303 through two meshing angle gears 307 and 308. The two meshing angle gears 307 and 308 constitute an angle gear driving mechanism. Thus, the cable 303 may also be driven to move by rotating the driver knob 305 so as to control the motion (e.g., a rotational motion) of the LITT probe (or the integrated probe 224). By using the meshing angle gears 307 and 308, the torque may be increased and transmitted, so that a relatively small driving moment applied on the driving knob 305 may drive the cable 303 to move. Similarly, the driving knob 306 is connected to the cable 302 through two meshing angle gears. In some embodiments, the cable 303 and 302 may be driven to move by controlling the driver knob 305 and 306 manually, thereby controlling the LITT probe (or the integrated probe 224) to move, which serves as a supplement to the manner of motor driving, and has a better flexibility.

FIGS. 4A-4C are schematic diagrams of an exemplary translation control mechanism and/or rotation control mechanism according to some embodiments of present disclosure.

The driving force output end 304 under different views may be shown in the figures. The driving force output end 304 includes a translation control mechanism and a rotation control mechanism. The translation control mechanism and the rotation control mechanism directly control the translational motion and rotational motion of the LITT probe (or the integrated probe 224), respectively.

Illustratively, the rotation control mechanism includes a synchronous belt drive assembly 403. The synchronous belt drive assembly 403 is connected to an LITT probe channel 404 and a rotation cable. The LITT probe (or integrated probe 224) is fixedly set in the LITT probe channel 404. As shown in FIG. 4A, a displacement of the rotation cable drives the synchronous belt drive assembly 403 to rotate via transformation of one or more intermediate component (e.g., an angle gear assembly, a worm and gear assembly), so that the LITT probe (or the integrated probe 224) may rotate. In this way, the synchronous belt drive assembly 403 implements the motion control of the LITT probe (or the integrated probe 224) at a certain rotating speed via a specific speed reduction ratio.

The translation control mechanism includes a worm and gear assembly 401 and a synchronous belt drive assembly 402. As shown in FIGS. 4B and 4C, a displacement of the translation cable is transmitted to the synchronous belt drive assembly 402 via the worm and gear assembly 401, which changes a direction of an input shaft, so that the LITT probe (or the integrated probe 224) may translate. Besides, a self-locking effect may be achieved, thereby improving a stability of the LITT probe (or the integrated probe 224) and eliminating or reducing influences of external forces. In some embodiments, the translation control mechanism and the rotation control mechanism may also be implemented by gear set transmission assemblies.

FIG. 5 is an exemplary structural diagram of the control device according to some embodiments of present disclosure.

As shown in the figure, the control device 208 includes an OCT imaging control module 501, an LITT treatment control module 502, a temperature measurement element control module 503, a head coil imaging registration module 504, a cooling system control module 505, an LITT probe position sensing and control module 506, a driving control module 507, and a supporting and fixing platform control module 508.

In combination with FIGS. 2A-2D, the OCT imaging control module 501 may control the OCT imaging of the target object. The OCT imaging control module 501 may control the OCT device to emit light signals, set one or more imaging parameters (e.g., a bandwidth of a light signal, a central wavelength of a light signal, an imaging duration, an image contrast, etc.) by generating corresponding instructions. In some embodiments, the OCT imaging control module 501 may also obtain pathological diagnostic signals, and generate one or more OCT images. The light signals emitted by the OCT device are transmitted to the OCT probe through the fiber optic slip ring 212.

The LITT treatment control module 502 may control the LITT probe to treat the target object. In some embodiments, the LITT treatment control module 502 integrates a laser controller, a tunable laser diode and/or an untunable laser diode. The laser controller may control the laser device (for example, the tunable laser diode and/or the untunable laser diode) to emit laser, so as to provide an ablation energy source for the LITT. The LITT treatment control module 502 may control the laser device to emit laser. The emitted laser passes through the optical relaying and processing device 211, and transmits to the LITT probe.

The temperature measurement element control module 503 may control the temperature measurement element to obtain temperature(s) of the target object and tissue around the target object and/or LITT probe. Temperature measurement signals of the temperature measurement element (such as heat source optical signals of the FBG sensor or analog temperature control signals of the K-type thermocouple) may be transmitted through the optical relaying and processing device 211.

The head coil imaging registration module 504 may control a registration between an image generated using the head coil (such as a head MRI image, a head thermal image) and an image generated using the MRI device (such as an MRI image, a thermal image), such that a clearer and more accurate image with temperature information may be obtained.

The cooling system control module 505 may control the cooling device for cooling the LITT probe. The cooling system control module 505 may control an on-off of the cooling source and parameters of the cooling medium (for example, a flow rate and a pressure of the CO₂ gas) through the cooling control element 213, and control the temperature of the LITT probe in combination with the temperature of the LITT probe measured by the temperature measurement element so as to prevent the probe from overheated and damaged, and protect the probe. In some embodiments, a temperature of health tissue around the target object may also be controlled in combination with temperature(s) of the target object and the tissue around the target object measured by the temperature measurement element so as to avoid unnecessary damages. In addition, the cooling system control module 505 may control a recycling of the cooling media in the cooling channel.

The LITT probe position sensing and control module 506 may obtain a real-time position of the LITT probe in the brain by controlling the LITT probe motion controller 210. The LITT probe position sensing and control module 506 may also send control signals to the LITT probe motion controller 210 in real time to issue a motion triggering instruction and receive a position feedback processing signal. The motion triggering instruction is used to trigger the LITT probe motion controller 210 to control the movement of the LITT probe. The position feedback processing signal is used to judge and determine a subsequent motion of the LITT probe (for example, continuing to move forward, adjusting a moving direction, retreating, etc.) based on the real-time position of the LITT probe.

The driving control module 507 may control the driving motor of the driving device 204 to drive the translation cable 215 and/or the rotation cable 216 to control the translational and/or rotational motion of the LITT probe (or the integrated probe 224) through the translation control mechanism 217 and the rotation control mechanism 218, respectively, thereby realizing an accurate motion control of the LITT probe (or the integrated probe 224).

The supporting and fixing platform control module 508 may control the motion of supporting and fixing platform 201. The supporting and fixing platform control module 508 controls the motion (e.g., translation, tilt, rotation, etc.) of supporting and fixing platform 201 by issuing motion control instructions, such that a position and posture of the patient may be adjusted, and a better baseline of scanning positions in MRI may be provided.

FIG. 6 illustrates two exemplary integrated probes of different types according to some embodiments of present disclosure.

As shown in FIG. 6, the integrated probe 224 connects the OCT probe channel, the LITT probe channel, the cooling channel, and the control cable channel of the temperature measurement element. The four channels set forth above are accommodated in the integrating channel of the integrating element 207 after being mechanically coupled by the integrating element 207. The integrating channel is connected to the integrated probe 224 directly or through a connector. Illustratively, the integrating channel is connected to the integrated probe 224 through the connector 601. The connector 601 is configured on the interface platform 205.

The two integrated probes of different types may include an LITT lateral ablation integrated probe 610 and an LITT circumferential ablation integrated probe 660.

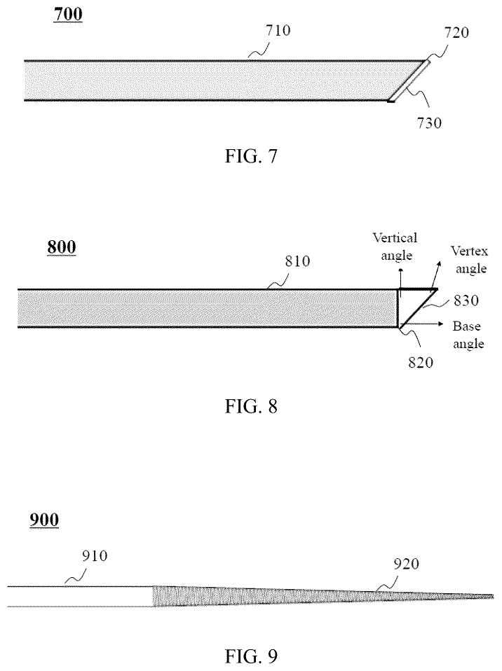

As shown in the figure, the LITT lateral ablation integrated probe 610 includes a temperature measurement element 611, a CO₂ feed channel 612, an LITT lateral ablation probe 613 (also referred to as LITT lateral ablation pin core), an OCT probe 614, a CO₂ collection channel 615. The LITT lateral ablation probe 613 of the LITT lateral ablation integrated probe 610 may be manufactured through at least two different preparation technologies. Detailed structures and preparation technologies of the LITT lateral ablation probe 613 may be referred to other figures (e.g., FIGS. 7 and 8) and the descriptions thereof.

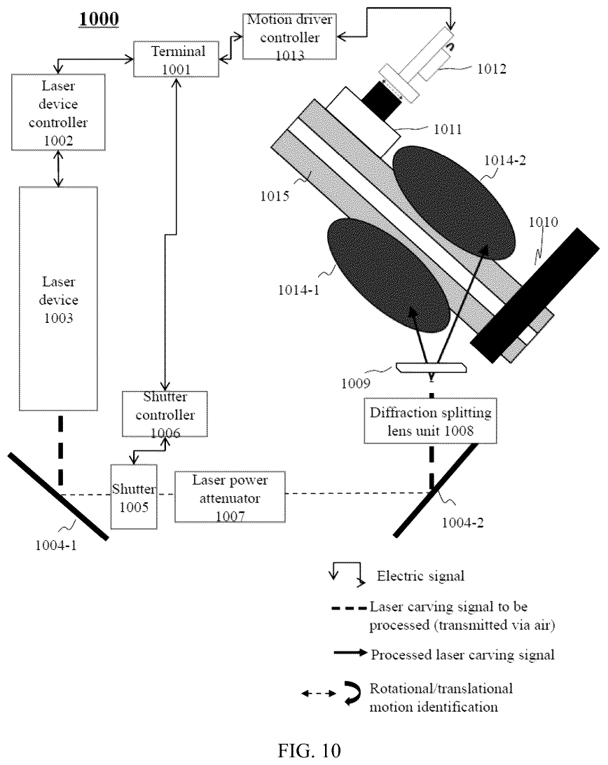

The LITT circumferential ablation integrated probe 660 includes a temperature measurement element 661, a CO₂ feed channel 662, an LITT circumferential ablation probe 663 (also known as LITT circumferential ablation pin core), an OCT probe 664, a CO₂ collection channel 665. Detailed structures and preparation technologies of the LITT circumferential ablation probe 663 may be referred to other figures (e.g., FIGS. 9-11) and the descriptions thereof.

FIG. 7 illustrates an exemplary LITT lateral ablation probe manufactured using a first preparation technology according to some embodiments of present disclosure.

As shown in FIG. 7, the LITT lateral ablation probe 700 includes a probe main body 710, a connection surface 720, and a coating layer 730. The connection surface 720 is a joint interface between the probe main body 710 and the coating layer 730.

The probe main body 710 has a shape of a cylinder. In some embodiments, an end (far end) of the probe main body 710 has an angle of inclination (i.e., an end surface of the probe main body 710 and an axis of the probe main body 710 form a certain angle, which ranges from 0 to 90°, excluding the two terminal points of the range). The probe main body 710 includes a probe core and a hard cladding structure located at an outer peripheral of the probe core. The probe core is made of pure silicon oxide material. The hard cladding structure is made of technology enhanced clad silica (TECS) material.

A diameter (outer diameter) of the probe core is, for example, 550-650 μm. In some embodiments, the diameter (outer diameter) of the probe core is 560 μm. In some embodiments, the diameter (outer diameter) of the probe core is 570 μm. In some embodiments, the diameter (outer diameter) of the probe core is 580 μm. In some embodiments, the diameter (outer diameter) of the probe core is 590 μm. In some embodiments, the diameter (outer diameter) of the probe core is 600 μm. In some embodiments, the diameter (outer diameter) of the probe core is 610 μm. In some embodiments, the diameter (outer diameter) of the probe core is 620 μm. In some embodiments, the diameter (outer diameter) of the probe core is 630 μm. In some embodiments, the diameter (outer diameter) of the probe core is 640 μm.

A thickness of the hard cladding structure is, for example, 5-30 μm. In some embodiments, the thickness of the hard cladding structure is 5 μm. In some embodiments, the thickness of the hard cladding structure is 10 μm. In some embodiments, the thickness of the hard cladding structure is 15 μm. In some embodiments, the thickness of the hard cladding structure is 20 μm. In some embodiments, the thickness of the hard cladding structure is 25 μm. In some embodiments, the thickness of the hard cladding structure is 30 μm.

The connection surface 720 is processed by removing a special step-index multimode fiber core of a certain numerical aperture (e.g., 0.35 NA, 0.36 NA, 0.37 NA, 0.39 NA, 0.4 NA, 0.41 NA, 0.42 NA, 0.43 NA, 0.44 NA, 0.45 NA, etc.) using a diamond membrane of a specific accuracy (e.g., 0.1 μm, 0.5 μm, 1 μm, 2 μm, 3 μm, etc.), setting a hard cladding structure of TECS at the outer peripheral as the main ingredient, and coating a suspension liquid of specific constituents on a surface of a basilemma of the colloidal silica, thus forming a brightened and cleaned total reflection lens surface.

Merely for illustration, the suspension liquid contains synthetic amorphous silica, H₂O, and propane-1,2-diol. In some embodiments, according to the mass fraction, the suspension liquid contains 10%-20% synthetic amorphous silica, 20%-30% H₂O, and 45%-55% propane-1,2-diol. In some embodiments, according to the mass fraction, the suspension liquid contains 20%-30% synthetic amorphous silica, 20%-30% H₂O, and 45%-55% propane-1,2-diol. In some embodiments, according to the mass fraction, the suspension liquid contains 30%-40% synthetic amorphous silica, 20%-30% H₂O, and 45%-55% propane-1,2-diol. In some embodiments, according to the mass fraction, the suspension liquid contains 20%-30% synthetic amorphous silica, 10%-20% H₂O, and 45%-55% propane-1,2-diol. In some embodiments, according to the mass fraction, the suspension liquid contains 20%-30% synthetic amorphous silica, 30%-40% H₂O, and 45%-55% propane-1,2-diol. In some embodiments, according to the mass fraction, the suspension liquid contains 20%-30% synthetic amorphous silica, 20%-30% H₂O, and 35%-45% propane-1,2-diol. In some embodiments, according to the mass fraction, the suspension liquid contains 20%-30% synthetic amorphous silica, 20%-30% H₂O, and 55%-65% propane-1,2-diol.

In some embodiments, the coating layer 730 is a coating layer of noble metal target material. The noble metal may include, for example, gold, silver, and metal of the platinum family. The coating layer 730 is formed through evaporation coating (for example, resistance heating evaporation coating, electron beam heating evaporation coating, inductive heating evaporation coating), sputtering coating (such as magnetron sputtering coating), or ion plating. In some embodiments, the coating layer 730 is formed by performing magnetron sputtering coating on the connection surface 720. For example, the coating layer 730 may be a dual-layer structure, a lower layer (being close to the connection surface 720) includes pure silver (e.g., 99.99%, 99.999%, 99.9999%, or 99.99999%) of a specific thickness (e.g., 60 nanometers (nm), 80 nm, 100 nm, 120 nm, 140 nm) formed by magnetron sputtering coating, and an upper layer (being away from the connection surface 720) is a silicon monoxide protective layer of a specific thickness (e.g., 120 nm, 130 nm, 140 nm, 150 nm, 160 nm, 180 nm) formed by magnetron sputtering coating. As another example, the coating layer 730 may be a single layer structure, which includes pure gold (e.g., 99.99%, 99.999%, 99.9999%, or 99.99999%) of a specific thickness (e.g., 60 nm, 80 nm, 100 nm, 120 nm, 140 nm) formed by magnetron sputtering coating.

In some embodiments, the coating layer 730 and the axis of the probe main body 710 forms a certain angle. The angle is, for example, 35°, 37°, 39°, 410, 43°, 45°, 47°, 49°, etc. The pure silver and silicon monoxide dual-layer structure mentioned above renders a reflectivity efficiency of 87% of the LITT high-power ablation laser in combination with the angle of the coating layer 730. In comparison with the application of total internal reflection (TIR, which requires that an incident angle on a polished surface exceeds a critical angle of about 43.5°) for light beam deflection, the coating layer of pure silver may prevent the light beam from leaking through the polished surface, which benefits a compatibility for a wider incident angle (below the angle range of the TIR) and a uniform focus point, and improves a reflective rate, a deflection performance of a light path, and a stability over the TIR.

By using the first preparation technology, the LITT lateral ablation probe 700 emits controllable laser, at an emission beam angle of about 81°, to the target object uniformly, such that an ablation of lesion tissue may be realized.

Correspondingly, the preparation technology (i.e., the first preparation technology) of the LITT lateral ablation probe 700 includes one or more of the following operations.

Step S7-1: the end surface of the main body 710 is brightened and cleaned.

The end surface of the main body 710 herein refers to the joint interface between the main body 710 and the coating layer 730, i.e., the connection surface 720. A special step-index multimode fiber core of a certain numerical aperture is removed using a diamond membrane of a specific accuracy. Then a hard cladding structure of TECS is set at the outer peripheral as the main ingredient. Finally, a suspension liquid of specific constituents is coated on a surface of a basilemma of the colloidal silica, thus rendering the end surface of the main body to form a brightened and cleaned surface. Illustratively, the suspension liquid contains synthetic amorphous silica, H₂O, and propane-1,2-diol. In some embodiments, according to the mass fraction, the suspension liquid contains 20%-30% synthetic amorphous silica, 20%-30% H₂O, and 45%-55% propane-1,2-diol.

It should be noted that the end (far end) of the probe main body 710 has a certain angle of inclination, i.e., the end surface of the probe main body 710 and the axis of the probe main body 710 form a certain angle. If the end of the probe main body 710 does not have an angle of inclination, i.e., the end surface of the probe main body 710 is perpendicular to the axis of the probe main body 710, a polishing operation may be performed. Via polishing, the end surface of the probe main body 710 and the axis of the probe main body 710 may form the certain angle, which ranges from 0 to 90°, excluding the two terminal points of the range. The end surface of the probe main body 710 may be polished via mechanical processing, a grinding wheel.

Step S7-2: the coating layer is covered on the brightened and cleaned end surface of the probe main body through magnetron sputtering coating, and the LITT lateral ablation probe 700 is formed.

The end surface of the main body 710 is coated using noble metal target material. In some embodiments, the coating layer 730 is formed by magnetron sputtering coating on the end surface of the main body 710. The coating layer 730 may be a dual-layer structure, a lower layer (being close to the connection surface) includes pure silver (e.g., 99.99%, 99.999%, 99.9999%, or 99.99999%) of a specific thickness (e.g., 60 nanometers (nm), 80 nm, 100 nm, 120 nm, 140 nm) formed by magnetron sputtering coating, and an upper layer (being away from the connection surface) is a silicon monoxide protective layer of a specific thickness (e.g., 120 nm, 130 nm, 140 nm, 150 nm, 160 nm, 180 nm) formed by magnetron sputtering coating. The coating layer 730 may also be a single layer structure, which includes pure gold (e.g., 99.99%, 99.999%, 99.9999%, or 99.99999%) of a specific thickness (e.g., 60 nm, 80 nm, 100 nm, 120 nm, 140 nm) formed by magnetron sputtering coating.

FIG. 8 illustrates an exemplary LITT lateral ablation probe manufactured using a second preparation technology according to some embodiments of present disclosure.

As shown in FIG. 8, the LITT lateral ablation probe 800 includes a probe main body 810, a connection surface 820, and a lens 830. The connection surface 820 is a joint interface between the probe main body 810 and the coating layer 830.

The probe main body 810 has a shape of a cylinder. The probe main body 810 includes a probe core and a hard cladding structure located at an outer peripheral of the probe core. The probe core is made of pure silicon oxide material. The hard cladding structure is made of TECS material.

A diameter (outer diameter) of the probe core is, for example, 550-650 μm. In some embodiments, the diameter (outer diameter) of the probe core is 560 μm. In some embodiments, the diameter (outer diameter) of the probe core is 570 μm. In some embodiments, the diameter (outer diameter) of the probe core is 580 μm. In some embodiments, the diameter (outer diameter) of the probe core is 590 μm. In some embodiments, the diameter (outer diameter) of the probe core is 600 μm. In some embodiments, the diameter (outer diameter) of the probe core is 610 μm. In some embodiments, the diameter (outer diameter) of the probe core is 620 μm. In some embodiments, the diameter (outer diameter) of the probe core is 630 μm. In some embodiments, the diameter (outer diameter) of the probe core is 640 μm.

A thickness of the hard cladding structure is, for example, 5-30 μm. In some embodiments, the thickness of the hard cladding structure is 5 μm. In some embodiments, the thickness of the hard cladding structure is 10 μm. In some embodiments, the thickness of the hard cladding structure is 15 μm. In some embodiments, the thickness of the hard cladding structure is 20 μm. In some embodiments, the thickness of the hard cladding structure is 25 μm. In some embodiments, the thickness of the hard cladding structure is 30 μm.

The connection surface 820 is processed by removing a special step-index multimode fiber core of a certain numerical aperture (e.g., 0.35 NA, 0.36 NA, 0.37 NA, 0.39 NA, 0.4 NA, 0.41 NA, 0.42 NA, 0.43 NA, 0.44 NA, 0.45 NA, etc.) using a diamond membrane of a specific accuracy (e.g., 0.1 μm, 0.5 μm, 1 μm, 2 μm, μ3 m, etc.), setting a hard cladding structure of TECS at the outer peripheral as the main ingredient, and coating a suspension liquid of specific constituents on a surface of a basilemma of the colloidal silica, thus forming a brightened and cleaned surface perpendicular to the end (flat end) of the probe main body 810.

Merely for illustration, the suspension liquid contains synthetic amorphous silica, H₂O, and propane-1,2-diol. In some embodiments, according to the mass fraction, the suspension liquid contains 10%-20% synthetic amorphous silica, 20%-30% H₂O, and 45%-55% propane-1,2-diol. In some embodiments, according to the mass fraction, the suspension liquid contains 20%-30% synthetic amorphous silica, 20%-30% H₂O, and 45%-55% propane-1,2-diol. In some embodiments, according to the mass fraction, the suspension liquid contains 30%-40% synthetic amorphous silica, 20%-30% H₂O, and 45%-55% propane-1,2-diol. In some embodiments, according to the mass fraction, the suspension liquid contains 20%-30% synthetic amorphous silica, 10%-20% H₂O, and 45%-55% propane-1,2-diol. In some embodiments, according to the mass fraction, the suspension liquid contains 20%-30% synthetic amorphous silica, 30%-40% H₂O, and 45%-55% propane-1,2-diol. In some embodiments, according to the mass fraction, the suspension liquid contains 20%-30% synthetic amorphous silica, 20%-30% H₂O, and 35%-45% propane-1,2-diol. In some embodiments, according to the mass fraction, the suspension liquid contains 20%-30% synthetic amorphous silica, 20%-30% H₂O, and 55%-65% propane-1,2-diol.

In some embodiments, the lens 830 is a sapphire lens. As shown in the figure, the lens 830 includes a vertical angle (90°), a base angle, and a vertex angle. A value of the vertex angle may be determined according to a value of the base angle. The vertical angle, base angle, and the vertex angle meet a certain relationship, such as the value of the vertex angle is equal to 90° minus the value of the base angle. It should be noted that in this embodiment, the main body 810 and the lens 830 are cylinders, and the lens 830 is a chamfered cylindrical lens. The vertical angle, the base angle, and the vertex angle herein are angles formed by different sides of the lens 830 in a two-dimensional image formed by viewing the lens 830 in a radial direction parallel to the chamfered surface.

The base angle is within a certain angle range. In some embodiments, the base angle is 50°-45°. Correspondingly, the vertex angle is 40°-45°. In some embodiments, the base angle is 52°-47°. Correspondingly, the vertex angle is 38°-43°. In some embodiments, the base angle is 54°-49°. Correspondingly, the vertex angle is 36°-41°. In some embodiments, the base angle is 56°-51°. Correspondingly, the vertex angle is 34°-39°.

A length of a cathetus between the base angle and the vertical angle of the lens 830 (i.e., the cathetus forming the base angle), i.e., the diameter of the cylindrical lens, is equal to the outer diameter of the probe main body 810, i.e., the outer diameter of the hard cladding structure made of TECS.

The connection surface 820 and a surface of the lens 830 where the cathetus between the base angle and the vertical angle is located is connected through fusion welding using electric arc welding or melted tungsten (such as tungsten wires), iridium (such as iridium wires), etc., at a high temperature. As for the fusion welding using the melted tungsten or iridium at the high temperature, a fire polishing processing may be performed on the tungsten or iridium, i.e., a welded surface between the probe main body and the lens is processed using the fire polishing, and the LITT lateral ablation probe 800 is formed eventually. The fire polishing is also referred to as flame polishing, may be performed using a flame polishing machine. A combination of such a processing technology and the vertex angle of the lens, which is in a specific angle range (e.g., 40°-45°, 38°-43°, 36°-41°, 34°-39°) promotes a performance of the LITT lateral ablation probe 800.

By using the second preparation technology, the LITT lateral ablation probe 800 emits controllable laser, at an emission beam angle of about 78°, to the target object uniformly, such that an ablation of lesion tissue may be realized.

In some embodiments, the lens 830 may also be a chamfered hemi-spherical lens (such as a sapphire hemi-spherical lens). A diameter of the chamfered hemi-spherical lens is, for example, 0.5 mm, 0.6 mm, 0.7 mm, 0.8 mm, 0.9 mm, 1 mm, etc. In some embodiments, the lens 830 may also be a chamfered hemi-ellipsoidal lens (such as sapphire hemi-ellipsoidal lens). A semi-major axis of the chamfered hemi-ellipsoidal lens is, for example, 0.5 mm, 0.6 mm, 0.7 mm, 0.8 mm, 0.9 mm, 1 mm, 1.1 mm, 1.2 mm, etc. An oblateness of the chamfered hemi-ellipsoidal lens is, for example, 1/298, 1/250, 1/200, 1/150, 1/100, 1/50, etc.

Correspondingly, the preparation technology (i.e., the second preparation technology) of the LITT lateral ablation probe 800 includes one or more of the following operations.

Step S8-1: an end surface of the main body 810 is brightened and cleaned.

The end surface of the main body 810 herein refers to the joint interface between the main body 810 and the lens 830, i.e., the connection surface 820. The end surface of the main body 810 is perpendicular to an axis of the main body 810. A special step-index multimode fiber core of a certain numerical aperture (e.g., 0.35 NA, 0.36 NA, 0.37 NA, 0.39 NA, 0.4 NA, 0.41 NA, 0.42 NA, 0.43 NA, 0.44 NA, 0.45 NA, etc.) is removed using a diamond membrane of a specific accuracy (e.g., 0.1 μm, 0.5 μm, 1 μm, 2 μm, 3 μm, etc.). Then a hard cladding structure of TECS is set at the outer peripheral as the main ingredient. Finally, a suspension liquid of specific constituents is coated on a surface of a basilemma of the colloidal silica, thus rendering the end surface of the probe main body 810 to form a brightened and cleaned surface. Illustratively, the suspension liquid contains synthetic amorphous silica, H₂O, and propane-1,2-diol. In some embodiments, according to the mass fraction, the suspension liquid contains 20%-30% synthetic amorphous silica, 20%-30% H₂O, and 45%-55% propane-1,2-diol.

Step S8-2: the main body 810 and lens 830 are connected through fusion welding to form the LITT lateral ablation probe 800.

A stable connection between the main body 810 and lens 830 are established through fusion welding using a specific high temperature processing technology. In some embodiments, the main body 810 and lens 830 may be fusion welded through electric arc welding. In some other embodiments, tungsten (such as tungsten wires), iridium (such as iridium wires), etc., is melted at a high temperature, and the main body 810 and lens 830 may be fusion welded using the melted tungsten, iridium, etc. and other materials may also be used to melt the main body 810 and lens 830. As for the fusion welding using the melted tungsten or iridium at the high temperature, a fire polishing processing may be performed on the tungsten or iridium, i.e., a welded surface between the probe main body and the lens is processed using the fire polishing, and the LITT lateral ablation probe 800 is formed eventually.

FIG. 9 illustrates an exemplary LITT circumferential ablation probe according to some embodiments of present disclosure.

As shown in FIG. 9, the LITT circumferential ablation probe 900 includes a probe main body 910.

The probe main 910 includes a probe core and a hard cladding structure located at an outer peripheral of the probe core. The probe core is made of pure silicon oxide material. The hard cladding structure is made of TECS material.

A diameter (outer diameter) of the probe core is, for example, 550-650 μm. In some embodiments, the diameter (outer diameter) of the probe core is 560 μm. In some embodiments, the diameter (outer diameter) of the probe core is 570 μm. In some embodiments, the diameter (outer diameter) of the probe core is 580 μm. In some embodiments, the diameter (outer diameter) of the probe core is 590 μm. In some embodiments, the diameter (outer diameter) of the probe core is 600 μm. In some embodiments, the diameter (outer diameter) of the probe core is 610 μm. In some embodiments, the diameter (outer diameter) of the probe core is 620 μm. In some embodiments, the diameter (outer diameter) of the probe core is 630 μm. In some embodiments, the diameter (outer diameter) of the probe core is 640 μm.

A thickness of the hard cladding structure is, for example, 5-30 μm. In some embodiments, the thickness of the hard cladding structure is 5 μm. In some embodiments, the thickness of the hard cladding structure is 10 μm. In some embodiments, the thickness of the hard cladding structure is 15 μm. In some embodiments, the thickness of the hard cladding structure is 20 μm. In some embodiments, the thickness of the hard cladding structure is 25 μm. In some embodiments, the thickness of the hard cladding structure is 30 μm.

In this embodiment, a far end (an end which is closer to the target object) of the probe main body 910 includes a cone-shaped surface 920. A diameter of the cone-shaped surface reduces from an initial diameter to a preset diameter gradually. The initial diameter may be a sum of a diameter of the probe core and twice of a thickness of the cladding structure as mentioned above. Illustratively, the initial diameter is 630 μm. The preset diameter is a diameter of the far end of the probe main body 910. The preset diameter is 50-150 μm. In some embodiments, the preset diameter is 60 μm. In some embodiments, the preset diameter is 70 μm. In some embodiments, the preset diameter is 80 μm. In some embodiments, the preset diameter is 90 μm. In some embodiments, the preset diameter is 100 μm. In some embodiments, the preset diameter is 110 μm. In some embodiments, the preset diameter is 120 μm. In some embodiments, the preset diameter is 130 μm. In some embodiments, the preset diameter is 140 μm.

One or more grooves are set on the cone-shaped surface 920. Laser transmitted to the LITT circumferential ablation probe 900 may be emitted out of the one or more grooves to treat the target object, thereby achieving ablation of lesion tissue. The one or more grooves are evenly (dispersively) distributed on the cone-shaped surface 920 in an arabesquitic pattern (e.g., a thread pattern), and do not overlap. The LITT circumferential ablation probe is also referred to as LITT circumferentially dispersive ablation probe. Each of the one or more grooves has a certain depth. Illustratively, the depth of the groove is 5-35 μm. In some embodiments, the depth of the groove is 10 μm. In some embodiments, the depth of the groove is 15 μm. In some embodiments, the depth of the groove is 20 μm. In some embodiments, the depth of the groove is 25 μm. In some embodiments, the depth of the groove is 30 μm.

In some embodiments, the arabesquitic pattern of the one or more grooves includes a single thread pattern (bostrychoid), a cross-thread pattern (e.g., dual-thread crossing pattern, as shown in FIG. 9), a rhombic grid pattern, a honeycomb pattern, etc., or a combination thereof. It should be noted that the single thread pattern, the cross-thread pattern, the rhombic grid pattern, the honeycomb pattern are merely specific examples, and not intended to limit specific arabesquitic pattern of the one or more grooves, which may be any suitable arabesquitic pattern that is evenly (dispersively) distributed along the cone-shaped surface 920, and does not overlap.

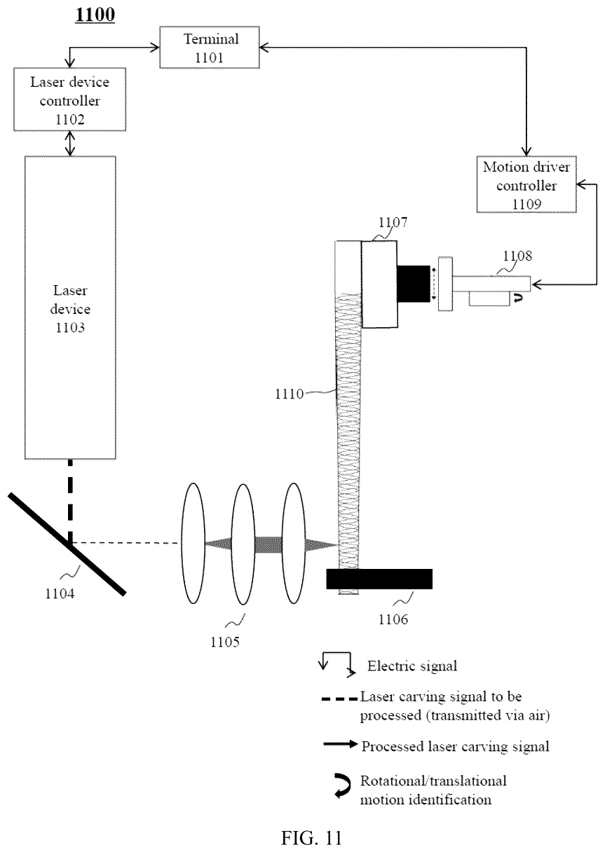

In some embodiments, the cone-shaped surface 920 and the one or more grooves that are evenly distributed on the cone-shaped surface 920 in the arabesquitic pattern may be carved using an optoelectronic device (e.g., an optical machine). Merely by way of example, the cone-shaped surface 920 and the one or more grooves that are evenly distributed on the cone-shaped surface 920 in the cross-thread pattern may be carved through optoelectronic devices and methods described in FIGS. 10 and 11, respectively. In some embodiments, the cone-shaped surface 920 and the one or more grooves set on the cone-shaped surface 920 may also be processed in other ways, such as mechanical processing.

FIG. 10 is a schematic diagram illustrating the processing of a cone-shaped surface according to some embodiments of present disclosure.

In this embodiment, the cone-shaped surface 920 is carved using a cone-shape carving device 1000. The cone-shape carving device 1000 includes a terminal 1001, a laser device controller 1002, a laser device 1003, reflecting mirrors 1004-1 and 1004-2, a shutter 1005, a shutter controller 1006, a laser power attenuator 1007, a diffraction splitting lens unit 1008, a focusing lens unit 1009, a sliding rail 1010, a fixing device 1011, a motion driver 1012, and a motion driver controller 1013.

The terminal 1001 is connected to and controls the laser device controller 1002, the shutter controller 1006, and the motion driver controller 1013. The laser device controller 1002 is connected to and controls the laser device 1003. The terminal 1101 may be, for example, a computer. The laser device 1003 may be a high-power CO2 continuous-wave laser device. The reflecting mirrors 1004-1 and 1004-2 are silver mirrors. The shutter 1005 may be an electric shutter. The shutter controller 1006 is connected to and controls a switch of the shutter 1005 so as to control the opening and closing of a laser path. The diffraction splitting lens unit 1008 and the focusing lens unit 1009 are used to split a laser beam into two laser beams and focusing the two laser beams, respectively. The focusing lens unit 1009 may be, for example, a zinc selenide lens. The motor driver controller 1013 is connected to and controls the motion driver 1012 to drive a first workpiece with cone-shaped surface to be carved 1015 with cone-shaped surface to be carved to move, such as translate and/or rotate.

The process for processing the first workpiece 1015 using the cone-shape carving device 1000 includes one or more of the following steps.

Step S10-1: two ends of the first workpiece 1015 are fixed on the sliding rail 1010 and the fixing device 1011, respectively. The fixing device 1011 is connected to the motion driver 1012. The fixing device 1011 may be a clamp. The clamp may fixedly grip one end of the first workpiece 1015.

Step S10-2: the laser device 1003 is controlled, by the laser device controller 1002 via the terminal 1001, to emit laser.

Laser generated by the laser device 1003 has a specific power and a specific wavelength. The power of the generated laser is, e.g., within a range of 20-40 W. In some embodiments, the power of the generated laser is 25 W. In some embodiments, the power of the generated laser is 30 W. In some embodiments, the power of the generated laser is 35 W. The wavelength of the generated laser is about 10600 nm. The laser is reflected to the shutter 1005 by the reflecting mirror 1004-1.

Step S10-3: the shutter 1005 is controlled, by the shutter controller 1006 via the terminal 1001, to be opened, and the laser is transmitted to the laser power attenuator 1007 for power attenuation processing.

The laser power attenuator 1007 attenuates a power of input laser at a certain percentage, for example, 10%, 20%, 30%, 40%, 50%, 60%, 70%, 90%, etc., that is, perform a power attenuation processing. A power of laser output from the laser power attenuator 1007 (i.e., attenuated power of the laser) is within a certain range, for example, 3-20 W. In some embodiments, the attenuated power of the laser is 6-15 W. In some embodiments, the attenuated power of the laser is 6.6-11.2 W. After the power attenuation processing, the laser is transmitted to the reflecting mirror 1004-2. The laser reflected by the reflecting mirror 1004-2 is transmitted to the diffraction splitting lens unit 1008. The diffraction splitting lens unit 1008 may be a beam splitting lens customized for the laser of the specific wavelength.

Step S10-4: the attenuated laser is split into a two laser beams by the diffraction splitting lens unit 1008, and the two laser beams are focused onto a surface of the first workpiece 1015 by the focusing lens unit 1009.

The first workpiece 1015 includes a probe core and a hard cladding structure located at an outer peripheral of the probe core. The probe core is made of pure silicon oxide material. The hard cladding structure is made of TECS material. The two laser beams form light spots 1014-1 and 1014-2 on the surface of the hard cladding structure of the first workpiece 1015.

Step S10-5: the first workpiece 1015 is driven to move by the motion driver 1012, which is controlled by the motion driver controller 1013 via the terminal 1001.

During the motion (translation, rotation, etc.) process of the first workpiece 1015, the light spots 1014-1 and 1014-2 formed by laser carve the surface of the first workpiece 1015, and the cone-shaped surface 920 is formed finally.

It should be noted that an order of the above steps is not limited in the present disclosure, and at the same time, a part of steps may also be incorporated and executed.

FIG. 11 is a schematic diagram illustrating the processing of one or more grooves distributed in a cross-thread pattern according to some embodiments of present disclosure.

In this embodiment, the one or more grooves distributed in the cross-thread pattern are carved on a surface of a second workpiece with arabesquitic pattern to be carved 1110 using an arabesquitic pattern carving device 1100. The second workpiece 1110 is a workpiece with a cone-shaped surface 920, which is obtained by performing the steps S10-1 through S10-5 as set forth above. The arabesquitic pattern carving device 1100 includes a terminal 1101, a laser device controller 1102, a laser device 1103, a reflecting mirror 1104, a lens group 1005, a sliding rail 1106, a fixing device 1107, a motion driver 1108, and a motion driver controller 1109.

The terminal 1101 is connected to and controls the laser device controller 1102 and the motion driver controller 1109. The terminal 1101 may be, for example, a computer. The laser device controller 1102 is connected to and controls the laser device 1103. The laser device 1103 may be a high-power CO₂ continuous-wave laser device. The reflecting mirrors 1104 may be a silver mirror. The lens group 1005 may include one or more lenses. The one or more lenses include at least one concave lens and/or at least one convex lens. In some embodiments, the motion driver controller 1109 is connected to and controls the motion driver 1108 to drive the second workpiece 1110 to move, for example, translate and/or rotate.

The process for processing the one or more grooves distributed in the cross-thread pattern using the arabesquitic pattern carving device 1100 includes one or more of the following steps.

Step S11-1: two ends of the second workpiece 1110 are fixed on the sliding rail 1106 and fixing devices 1107, respectively. The fixing device 1107 is connected to the motion driver 1112. The fixing device 1107 may be a clamp. The clamp may fixedly grip one end of the second workpiece 1110.

Step S11-2: the laser device 1103 is controlled, by the laser device controller 1102 via the terminal 1101, to emit laser.

Laser generated by the laser device 1103 has a specific power and a specific wavelength. The power of the generated laser is, e.g., within a range of 20-40 W. In some embodiments, the power of the generated laser is 25 W. In some embodiments, the power of the generated laser is 30 W. In some embodiments, the power of the generated laser is 35 W. The wavelength of the generated laser is about 10600 nm. In this embodiment, the laser device 1103 may be a high-power CO₂ continuous-wave laser device. As for continuous waves generated by the high-power CO₂ continuous-wave laser device, a damage threshold of the second workpiece 1110 is about 250 kW/cm²; as for pulse waves (for example, pulse waves of 10 ns) generated by the high-power CO₂ continuous-wave laser device, the damage threshold of the second workpiece 1110 is about 1 GW/cm².

Step S11-3: the laser is focused on a surface of the second workpiece 1110 by the lens group 1105.

The laser passes through the lens group 1105, and forms a light spot of specific energy and a specific size on the cone-shaped surface of the second workpiece 1110. For example, a diameter of the light spot is 30-60 μm. In some embodiments, the diameter of the light spot is m. In some embodiments, the diameter of the light spot is 40 μm. In some embodiments, the diameter of the light spot is 45 μm. In some embodiments, the diameter of the light spot is 50 μm. In some embodiments, the diameter of the light spot is 55 μm. Energy of the light spot is higher than an irradiance for melting of the surface material of the second workpiece 1110. The irradiance of the surface material of the second workpiece 1110 is close to an irradiance for melting glass, i.e., 3.1×10⁵ W/cm².

In some embodiments, the lens group 1105 may include a first concave lens, a first convex lens, and a second concave lens arranged in sequence. The first concave lens and the first convex lens are used to extend the diameter of the laser beam to a first diameter. The second concave lens is used to focus the laser beam of the first diameter to the surface of the second workpiece 1110 to form the light spot as mentioned above.

Step S11-4: the second workpiece 1110 is driven to move by the motion driver 1108, which is controlled by the motion driver controller 1109 via the terminal 1101.

During the motion (translation, rotation, etc.) process of the second workpiece 1110, the light spot formed by laser carves the surface of the second workpiece 1110, and the one or more grooves in the cross-thread pattern are formed on the cone-surface of the second workpiece 1110 finally.

In some embodiments, when the arabesquitic pattern carving device 1100 carves the one or more grooves in the cross-thread pattern, an air cooling system may be used to sweep the carved grooves so as to remove particles (e.g., dust, impurities), melts, etc., inside and around the grooves, and keep the surface of the processing area clean.

It should be noted that an order of the above steps is not limited in the present disclosure, and at the same time, a part of steps may also be incorporated and executed.

A distribution of vectorial optical energy of the LITT circumferential ablation probe 900 manufactured using the optoelectronic devices and the methods as described with reference to FIGS. 10 and 11 may be more uniform since the one or more grooves in the arabesquitic pattern (e.g., cross-thread pattern) are evenly (dispersively) distributed along the cone-shaped surface 920. In combination with the temperature measurement mechanism of the temperature measurement element as set forth above, ablation of surrounding tissue becomes more uniform and radical. In the meanwhile, combining with thermal characteristics of tissue and heat injury algorithms based on the LITT circumferential ablation probe 900, both a protection of healthy tissue in the treatment and ablation effect of ablation pathology may have remarkable improvements.

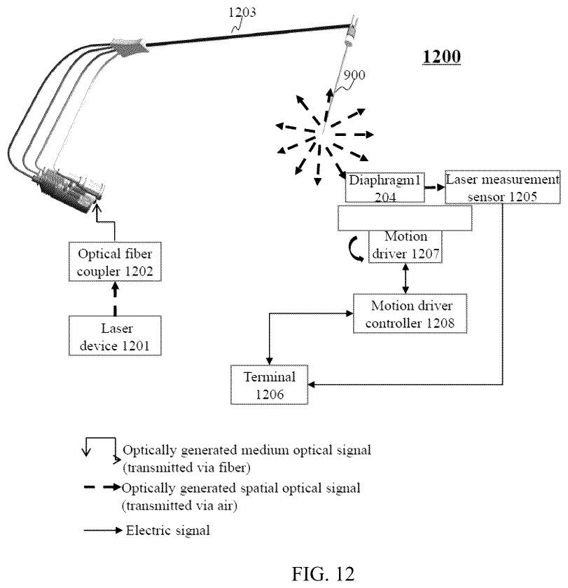

FIG. 12 is a schematic diagram illustrating a test of a distribution of vectorial optical energy of an LITT circumferential ablation probe according to some embodiments of the present disclosure.

In this embodiment, a distribution uniformity of the vectorial optical energy of the LITT circumferential ablation probe 900 may be tested using an LITT probe polarity testing device 1200. The distribution uniformity of the vectorial optical energy of the LITT circumferential ablation probe 900 may be characterized by polar intensities of light distribution of the LITT circumferential ablation probe 900 in circular spaces at different heights. The LITT probe polarity testing device 1200 includes a laser device 1201, an optical fiber coupler 1202, an LITT circumferential ablation integrated probe 1203, a diaphragm 1204, a laser measurement sensor 1205, a terminal 1206, a motion driver 1207, and a motion driver controller 1208.

The laser device 1201 may be a helium-neon laser device. In some embodiments, the helium-neon laser device may be a laser device that generates laser having a wavelength of 632.8 nm. The diaphragm 1204 is used to restrict the propagation of a laser beam. The diaphragm 1204 is set in front of the laser measurement sensor 1205 to confine laser received by the laser measurement sensor 1205. For example, the laser measurement sensor 1205 may merely receive laser propagating in a specific direction. The diaphragm 1204 may be an aperture diaphragm, a slit diaphragm, etc. In some embodiments, the diaphragm 1204 may be a slit diaphragm. A size of the slit diaphragm (i.e., a width of a slit of the slit diaphragm) is, for example, 0.1-0.5 mm. In some embodiments, the size of the slit diaphragm is 0.2 mm. In some embodiments, the size of the slit diaphragm is 0.3 mm. In some embodiments, the size of the slit diaphragm is 0.4 mm. The laser measurement sensor 1205 is used to receive laser, and generates corresponding electric signals for characterizing the intensity of received laser. In some embodiments, the laser measurement sensor 1205 may be a laser measurement sensor of a circular geometry photodiode sensor or a sensor of other types that may measure the intensity of laser. The terminal 1206 is connected to and controls the laser measurement sensor 1205 and the motion driver controller 1208. The terminal 1206 may be, for example, a computer. The diaphragm 1204 and laser measurement sensor 1205 are fixedly connected to the motion driver 1207. The motion driver controller 1208 is connected to and controls the motion driver 1207 to drive the diaphragm 1204 and laser measurement sensor 1205 to move, for example, translate and/or rotate.

The process for testing the distribution uniformity of the vectorial optical energy of the LITT circumferential ablation probe 900 using the LITT probe polarity testing device 1200 includes one or more of the following steps.

Step S12-1: signals output by the laser measurement sensor 1205 is obtained via the terminal 1206 to generate a polar intensity of a specific orientation of the laser emitted by the LITT circumferential ablation probe.

The laser device 1201 generates laser (e.g., visible light). The laser is transmitted to the optical fiber coupler 1202 through a spatial light path (such as an optical fiber). The optical fiber coupler 1202 is coupled to the LITT probe channel of the LITT circumferential ablation integrated probe. The laser is transmitted to the LITT circumferential ablation probe 900 through the LITT probe channel, and vectorial optical energy is dispersively emitted out of the LITT circumferential ablation probe 900 at 360 degrees through the one or more grooves uniformly distributed in a circumferential direction. The vectorial optical energy is restricted by the diaphragm 1204, and transmitted into the laser measurement sensor 1205. The laser measurement sensor 1205 receives the incident laser, and generates corresponding electric signals for characterizing an intensity of the received laser. The intensity is the polar intensity of a specific orientation in which the diaphragm 1204 and laser measuring sensor 1205 are located.

Step S12-2: a distribution of the intensity of the laser in the entire space (also referred to as spatial intensity distribution) of the laser emitted by the LITT circumferential ablation probe is determined by controlling, by the motion driver controller 1208 via the terminal 1206, the motion driver 1207 to drive the diaphragm 1204 and the laser measurement sensor 1205 to move along a circumferential direction and an axial direction of the cone-shape surface of the LITT circumferential ablation probe 900.

The entire space refers to a combination of the circumferential direction (360 degrees) and axial direction (an overall length) of the LITT circumferential ablation probe 900.

The diaphragm 1204 and the laser measurement sensor 1205 are fixedly connected to the motion driver 1207. The motion driver 1207, controlled by the motion driver controller 1208, rotates in a circle, thus driving the diaphragm 1204 and the laser measurement sensor 1205 to move circularly surrounding the LITT circumferential ablation probe 900. Polar intensities of the vectorial light generated by the LITT circumferential ablation probe 900 in a circumferential distribution are obtained according to the above step S12-1.

Then the motion drive 1207 drives the diaphragm 1204 and the laser measurement sensor 1205 to move such that positions (also referred to as heights) of the driver 1204 and the laser measurement sensor 1205 relative to lengthwise direction of the LITT circumferential ablation probe 900 are adjusted. Polar intensities of the vectorial light generated by the LITT circumferential ablation probe 900 in a circumferential distribution at different heights are obtained, and the spatial intensity distribution of the laser (the vectorial light) is obtained.

Tests were conducted using the method described in the above S12-1 and S12-2 on the LITT circumferential ablation probe 900 manufactured using the optoelectronic devices and the methods as described with reference to FIGS. 10 and 11. Results of the tests show that a distribution of the vectorial optical energy of the LITT circumferential ablation probe 900 is remarkably uniform. Therefore, the LITT circumferential ablation probe 900 has better performance and treatment effect.

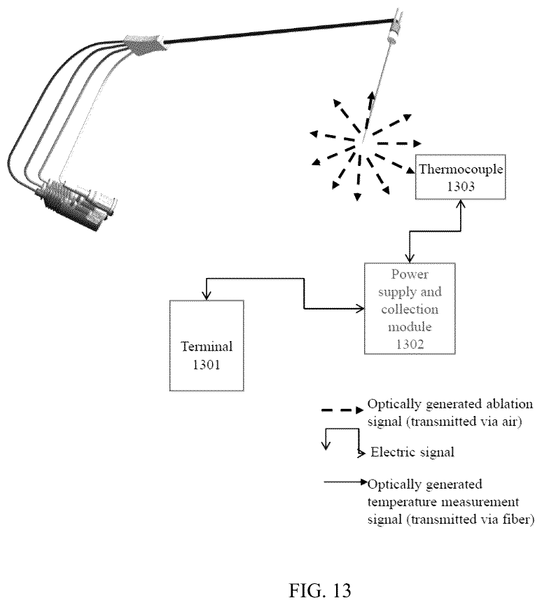

FIG. 13 is a schematic diagram illustrating temperature measurement of a thermocouple according to some embodiments of the present disclosure.

As set forth above, a temperature measurement element (temperature measurement element 611 or 661), such as a thermocouple 1303, may be set in the integrated probe (LITT lateral ablation integrated probe 610 or LITT circumferential ablation integrated probe 660) to measure temperature(s) of the LITT probe and/or the target object.

The thermocouple 1303 may be connected to a power supply and collection module 1302. The power supply and collection module 1302 further connects to a terminal 1301. The power supply and collection module 1302 provides a power supply for thermocouple 1303 and transmits the temperature measurement value(s) collected during a working process of the thermocouple 1303 to the terminal 1301. The terminal 1301 may be, for example, a computer.

The thermocouple 1303 may be a K-type thermocouple, a T-type thermocouple, an E-type thermocouple, etc. In this embodiment, the thermocouple 1303 is a K-type thermocouple. In some embodiments, parameters of the K-type thermocouple may include a diameter of 40-60 μm, a resistance value of 46-55 Ohm (Ω), and a measurement accuracy of ±0.5-2 degrees centigrade (° C.). Illustratively, the parameters of the K-type thermocouple are that the diameter is 50 μm, the resistance value is 51Ω, and the measurement accuracy is ±1.2° C.

When the LITT probe works, the thermocouple 1303 is positioned close to the target object. At the same time, the LITT probe produces laser ablation signals, which also affects the thermocouple 1303. The thermocouple 1303 is connected to the terminal 1301 via the power supply and collection module 1302, and obtains and records temperature variations of the target object and/or the LITT probe temperature change in real time. By measuring the temperature(s) of the target object and/or the LITT probe, since the thermocouple 1303 is closer to the tissue of the target object and the probe, temperature measurement results are more accurate than a remote temperature measurement of the MRTI.

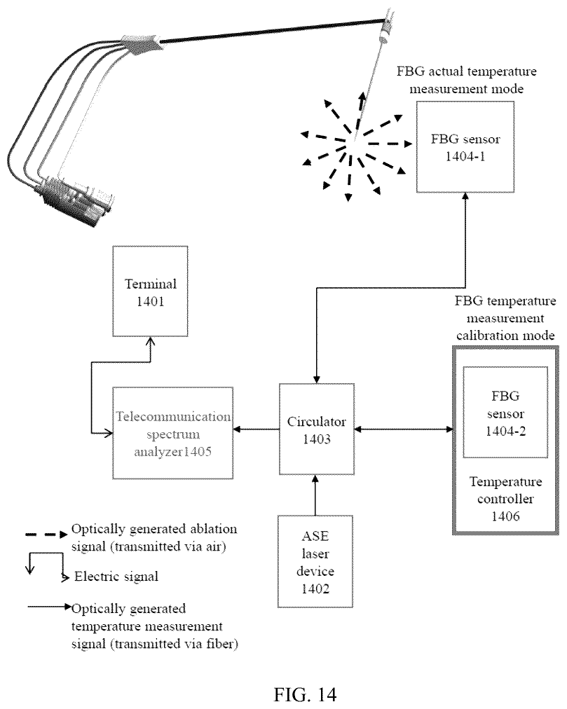

FIG. 14 is a schematic diagram illustrating temperature measurement of an FBG sensor according to some embodiments of the present disclosure.

As set forth above, a temperature measurement element (temperature measurement element 611 or 661), such as an FBG sensor 1404-1, may be set in the integrated probe (LITT lateral ablation integrated probe 610 or the LITT circumferential ablation integrated probe 660) to measure temperature(s) of the LITT probe and/or the target object.

During an actual application in temperature measurement under an FBG actual temperature measurement mode, the system includes a terminal 1401, an amplified spontaneous emission (ASE) laser device 1402, a circulator 1403, an FBG sensor 1404-1, a telecommunication spectrum analyzer 1405, and a temperature controller 1406. The terminal 1401 may be, for example, a computer. The ASE laser device 1402 has an ultra-broad band. For example, the ASE laser device 1402 may have S, C, and L bands three-in-one, an ultra-broad band of 140 nm, and a central wavelength of 1530 nm. The circulator 1403 may be a circulator having an ultra-broad band, multi-modes, and high power. The telecommunication spectrum analyzer 1405 is a telecommunication spectrum analyzer with an analysis range of 600-1700 nm.

A specific technique, for example, Multiphysics software is used to perform numerical calculations to predict a spatial temperature distribution and thermal denaturation of interstitial tissue in the brain during an LITT treatment preset by a doctor. A geometric structure for the simulation uses the LITT circumferential ablation probe 900 (as shown in the figure). A pipe sleeve made of poly-ether-ether-ketone (PEEK) or polycarbonate (PC) covering the main body 910 is connected to a glass cap (having a length of 40-60 mm and an outer diameter of 1.4 mm) at a far end of the LITT circumferential ablation probe 900. A brain tumor (tissue boundary) of 3 cm³ is set up. Tuned laser of 4 W and 980 nm emits through the LITT circumferential ablation probe 900 to ablate simulated brain tumor for 120S in a cylindrical shape (as shown in the dispersive directions in the figure).

ρ (kg/m³) is set as a tissue density, c (J/kg·K) is set as a tissue specific heat, K (W/m·K) is set as a tissue thermal conductivity, and T (° C.) is set as a tissue temperature. Since an in-vitro model is used, the impact of blood perfusion and metabolic heat production may be 0 actually; Qₗ(W/m³) is a laser induced heat source. In addition, an initial angle measurement confirms that an intensity of laser is transported in two directions: a radial transmission power of a diffusion part P₁ (89% of incident laser power in W) and a forward transmission power of a fiber tip P₂ (11% of the incident laser power in the W). Based on these settings, a thermal reaction of tissue during an LITT irradiation is quantified as:

$$ρ c \frac{\partial T ( r , t )}{\partial t} = ∇ \cdot [ k ∇ T ( r , t ) ] + {Q}_{t} , \qquad ( 1 )$$

where μₐ(cm⁻¹) denotes tissue absorption coefficient, μₛ(cm⁻¹) denotes tissue scattering coefficient, r(m) denotes the radial distance from a surface of a diffuser, and l(m) denotes a length of the diffuser. Based on a beam divergence angle (NA=0.5) at a tip of the diffuser, a spatial beam intensity in a radial direction is assumed to be a cylinder along an axis of the diffusion applicator, and a corresponding thermal source is quantified as:

$${Q}_{l , radical} = {μ}_{a} \cdot \frac{{P}_{1}}{2 π rl} {e}^{- ( {μ}_{a} + {μ}_{s} ) r} , \qquad ( 2 )$$

where σ (μm) denotes the size of a light spot of the laser beam, and z(m) denotes an axial depth in the tissue.

Assuming that forward beam distribution is Gaussian distribution, the thermal source is converted and quantified as:

$${Q}_{l , forward} = {μ}_{a} \frac{{P}_{2}}{{πσ}^{2}} {e}^{- \frac{{r}^{2}}{[ 2 {σ}^{2} exp ( {μ}_{s} z ) ]}} \cdot {e}^{- ( {μ}_{a} + {μ}_{s} ) z} , \qquad ( 3 )$$

therefore, the application of a wavelength of 980 nm or 1064 nm causes absorption effect and scattering effect to be more significant. An average cosine of a scattering function of thermal characteristics of tissue g is introduced. Therefore, an effective attenuation coefficient may replace the absorption coefficient:

μ_{eff}=\sqrt{3μₐ[μₐ+μₛ(1−g)]}. (4)

An initial temperature of the entire tissue is set to 20° C., and an insulation treatment is performed on an exterior tissue surface (that is [symbol] .k∇T=0, where [symbol] denotes the direction of heat flow). In addition, a degree of a heat damage is determined by using the Arrhenius parameter, mainly because a temperature dependence of the molecular reaction rate. As a first-order rate process, an Arrhenius damage integral is used to describe tissue heat damage (where Ω represents a dimensionless factor that defines an irreversible heat damage, A_{f}(1/s) represents a frequency factor, Eₐ (J/mol) represents degeneration activation energy, R represents general gas constant 8.314 (J/mol·K), and τ (s) represents LITT radiation time. Therefore, a start of an irreversible heat degeneration corresponds to a temperature of 60° C.-65° C. of the interstitial tissue, where Ω=1.

TABLE 1
Main parameters or constants of optical properties of a target organ
Main parameters or constants of optical properties of a target organ

| Parameter | Symbol, unit |
|---|---|
| Absorption coefficient | μₐ, 1/cm |
| Scattering coefficient | μₛ, 1/cm |
| Frequency factor | A_{f}, 1/s |
| Activation energy | Eₐ, J/mol |
| General gas constant | R, J/mol · K |

Table 1 shows main parameters or constants of optical properties of a target organ at 980 nm or 1064 nm. It is assumed that the parameters or constants remain constant during the LITT treatment:

$$Ω ( r , t ) = {A}_{f} \cdot {\int }_{0}^{τ} exp [ \frac{- {E}_{a}}{RT ( r , t )} ] dt , \qquad ( 5 )$$

Λ and n_{eff} are set as a grating period and an effective refractive index of a basic mode at a free space wavelength, respectively. λ_{B} is affected by a temperature variation of a periodical thermal expansion or shrinkage of the grid and a thermos-optic effect (thermal induction variation of n_{eff}). Thus, the FBG is used as a temperature sensing element. The FBG sensor 1404-1 is obtained in combination with the manufacturing process of the material of the FBG, which renders that the refractive index of the fiber core is modulated periodically. The periodic core index modulations generate the core modes. These modes reflect or transmit through index boundaries and interfere with each other. In turn, input light is strongly reflected merely at a specific wavelength determined by a certain phase matching condition. The specific wavelength is referred to as a Bragg wavelength (λ_{B}) of FBG. The phase matching condition is also referred to as a Bragg condition. λ_{B} is quantified as:

λ_{B}=2Λn_{eff}. (6)

ΔT=T_{H}−T₀. T₀ and T_{H} represent a reference and a high temperature applied to the FBG, respectively. λ_{B0} is the Bragg wavelength of the FBG at T₀. α_{Λ} and αₙ represents a thermal expansion coefficient and a thermal optical coefficient of a single pole fiber for manufacturing the FBG, respectively. A Bragg wavelength drift Δλ_{B} induced by the temperature variation ΔT is quantified as:

$$Δλ - B = 2 ( Λ \frac{\partial {n}_{eff}}{\partial T} + {n}_{eff} \frac{\partial Λ}{\partial T} ) Δ T = {λ}_{B 0} ( {α}_{n} + {α}_{Λ} ) Δ T , \qquad ( 7 )$$

By using a quantitatively pre-defined heat sensitivity S, a measurement of Δλ_{B} will enable a quantification of ΔT, i.e., an ablation temperature variation. According to the above formula (6), the heat sensitivity S of the FBG is quantified as:

$${S}_{FBG} = \frac{{Δλ}_{B}}{Δ T} = {λ}_{B 0} ( {α}_{Λ} + {α}_{n} ) . \qquad ( 8 )$$

Therefore, according to the algorithms as set forth above, the FBG sensor 1404-1 may monitor the temperature of the interstitial tissue during the LITT laser radiation in real time.

Before the FBG sensor 1404-1 is applied in the LITT probe for a treatment or auxiliary surgery, it is needed to obtain the heat sensitivity of the FBG in advance according to the formula (8), and calibrate a relationship between Δλ_{B} and ΔT based on the principle of the formula (7).

The calibration needs to be performed as a static calibration under a FBG temperature measurement calibration mode within a temperature range (for example, −50° C. to 180° C., −40° C. to 150° C., −30° C. to 120° C., −20° C. to 100° C.) controlled by the temperature controller 1406. The system for the calibration includes the terminal 1401, the ASE laser device 1402, the circulator 1403, an FBG sensor 1404-2, the telecommunications spectrum analyzer 1405, and the temperature controller 1406.

In the FBG temperature measurement calibration mode, the FBG sensor 1404-2 is placed in the temperature controller 1406, which controls the temperature in a range −40° C. to 150° C. The ASE laser device 1402 may be an ultra-broad band ASE laser device, and broadband light generated by the ASE laser device arrives the FBG sensor 1404-2 through the circulator 1403. Then a reflection signal of the FBG sensor 1404-2 accesses the telecommunication spectrum analyzer 1405 via the circulator 1403. The telecommunications spectrum analyzer 1405 is used to monitor a reflection spectrum of the FBG sensor 1404-2. Specifically, calibration examinations are performed within a specific temperature range (for example, 10° C.-80° C., 20° C.-100° C., 30° C.-120° C.). A temperature in the temperature controller changes periodically. An interval between two neighboring temperatures is 10° C. The entire process lasts for 5 hours. In this case, a dynamic Bragg wavelength drift Δλ_{B} corresponds to a temperature variation ΔT in the temperature controller 1406. According to another algorithm S_{FBG}=Δλ_{B}/ΔT, it is measured that S_{FBG}≈0.0114 nm/° C., i.e., the heat sensitivity S_{FBG} of the FBG obtained in advance. According to the formula (8), an actual temperature may be obtained by the terminal 1401 through Δλ_{B}.

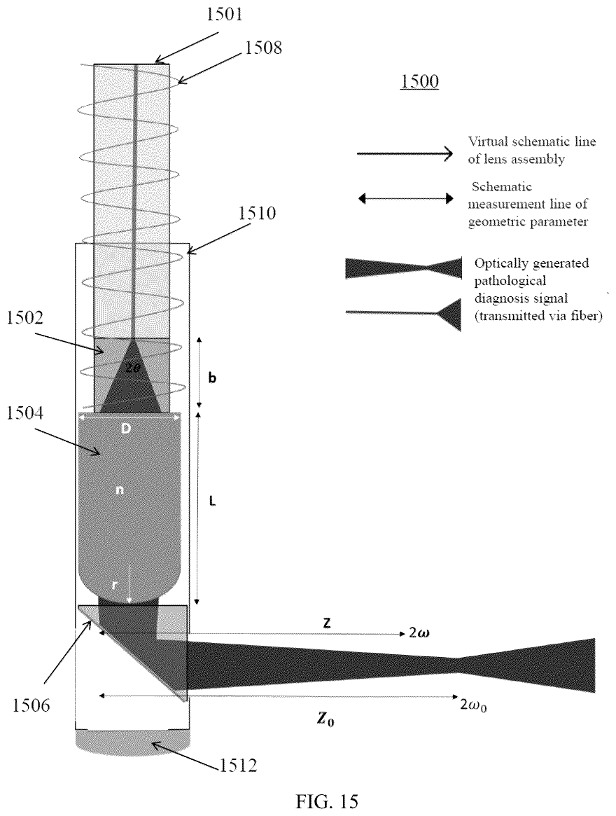

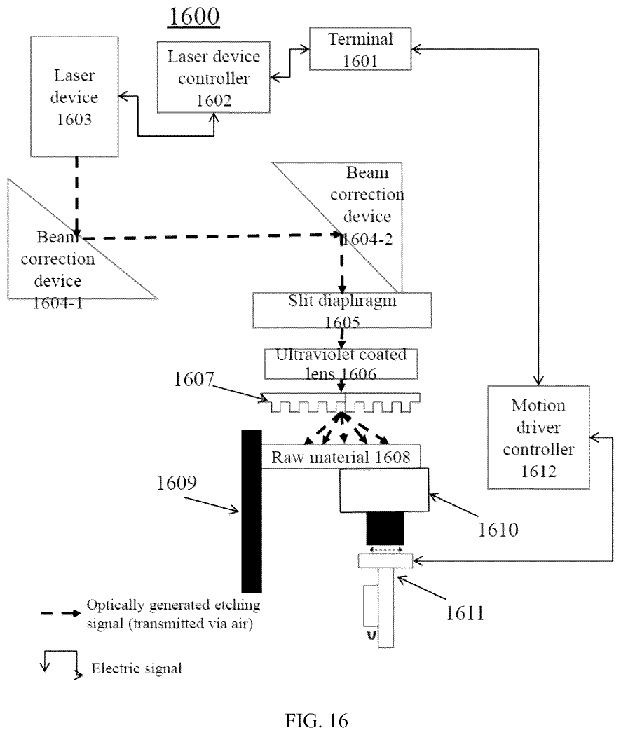

FIG. 15 illustrates an exemplary OCT probe according to some embodiments of the present disclosure.

The OCT probe 1500 has along working distance, which is suitable for assisting LITT ablation of the target object (e.g., cancer tissue) of a large size.

As shown in the figure, the OCT probe 1500 includes an input port 1501, a first lens 1502, a second lens 1504, a beam deflection unit 1506, a spring torsion coil 1508, an optical sleeve 1510 and a filler 1512. Two ends of the second lens 1504 are connected to the first lens 1502 and the beam deflection unit 1506, respectively, via fusion welding, which uses tungsten or iridium melted at the high temperature, and fire polishing, and two welded surfaces are formed, thereby facilitating fixed connections between the second lens 1504 and the first lens 1502 and the beam deflection unit 1506.

A light beam from the light source of the OCT device passes through a specific optical path, e.g., the fiber optic slip ring 212, the input port 1501 (e.g., in the form of a single mode fiber), and access the OCT probe 1500. The input port 1501 is configured to input the light beam into the OCT probe 1500. The light beam accessing the OCT probe 1500 passes through the first lens 1502, and the second lens 1504 sequentially, and emits out of the OCT probe 1500 after being deflected by the beam deflection unit 1506. The light beam existing the OCT probe 1500 may be used to irradiate the target object.

The first lens 1502 is configured to expand the light beam accessing the OCT probe 1500 (the first lens 1502). For example, the incident beam is a parallel beam, and the first lens 1502 may expand the parallel beam. The expanded beam has a specific divergence angle (also referred as expanded beam angle). The first lens 1502 has a shape of a cylinder.

In some embodiments, the first lens 1502 is a coreless lens. The expanded beam angle of the coreless lens is 2θ, where θ is, for example, 5°, 10°, 15°, 20°, 25°, etc. The coreless lens has a specific length b. In some embodiments, a focal length and a size of a focal spot of the OCT probe 1500 may relate to the length b of the coreless lens.

The second lens 1504 is configured at a posterior stage (rear end) of the first lens 1502. The light beam exiting the first lens 1502 accesses the second lens 1504. The second lens 1504 is configured to focus the light beam exiting the first lens 1502, and form an emergent light spectrum with a certain focal length. In some embodiments, the second lens 1504 may also reduce dispersion of the light beam exiting the first lens 1502.

In some embodiments, the second lens 1504 may be a micro plano-convex cylindrical lens. The micro plano-convex cylindrical lens has a start terminal and an end terminal in an axial direction (e.g., a direction along which the light beam transmits in the figure). As used herein, the start terminal refers to an end surface of the micro plano-convex cylindrical lens where the light beam accesses, and the end terminal refers to an end surface of the micro plano-convex cylindrical lens where the light beam exits. As shown in the figure, the start terminal of the micro plano-convex cylindrical lens is a planar surface, and the end terminal of the micro plano-convex cylindrical lens is a convex spherical surface. A body of the micro plano-convex cylindrical lens between the start terminal and the end terminal has a shape of a cylinder. The cylindrical body has a specific cross-sectional diameter (i.e., a diameter of a cross section of the cylindrical body). The cross-sectional diameter is, for example, 500 μm, 520 μm, 540 μm, 560 μm, 580 μm, 600 μm, 620 μm, etc. In some embodiments, the cross-sectional diameter is 560 μm, which may ensure the completeness of the light beam when passing through the micro plano-convex cylindrical lens. A cross-sectional diameter exceeding or being below 560 μm may damage the performance of the optical path (e.g., a cross-sectional diameter being below 560 μm may result in a failure of a mechanical compatibility between the optical path and the OCT probe 1500, and severe insertion and imaging bandwidth losses). An optical curvature r of the convex spherical surface at the end terminal of the micro plano-convex cylindrical lens is, for example, −1.5 mm, −1.6 mm, −1.7 mm, −1.8 mm, −1.9 mm, −2 mm, −2.1 mm, etc. In some embodiments, the optical curvature r of the convex spherical surface is −1.8 mm. The optical curvature of −1.8 mm plays a pivotal role in ensuring an effective working distance over 1 centimeter of the OCT probe 1500. Otherwise, the focal length of the OCT probe 1500 may shrink such that the working distance may be reduced, the focal spot may be enlarged, a lateral resolution may be reduced, and imaging quality may be lowered.

The material of the micro plano-convex cylindrical lens includes, for example, N-LAF3, SF11, N-SF11, or other optical materials. In some embodiments, the material of the micro plano-convex cylindrical lens is N-LAF3. The micro plano-convex cylindrical lens has a certain refractive index n. The refractive index n is, for example, 1.3, 1.4, 1.5, 1.6, 1.7, 1.8, etc.

In some embodiments, the start terminal (i.e., a beam incident end) of the second lens 1504 is polished, and has a certain angle. As used herein, the angle of the start terminal is an angle between a vertical plane of an axis of the second lens 1504 and the surface of the start terminal. The angle is, for example, 0°, 2°, 4°, 6°, 8°, 10°, etc. In some embodiments, the angle is 0° or 8°. The angle of 0° may enhance mechanical stress performance of a fusion welded surface after a fire polishing processing; the angle of 8° may reduce a TIR pollution value of the fusion welded surface dramatically, and reduce an insertion loss.

The beam deflection unit 1506 is configured at a posterior stage (rear end) of the second lens 1504. The light beam exiting the second lens 1504 accesses the beam deflection unit 1506. The beam deflection unit 1506 is configured to deflect the light beam exiting the second lens 1504. The deflected light beam exits the OCT probe 1500.

In some embodiments, the beam deflection unit 1506 includes a cylindrical fiber core and a hard cladding structure located at an outer peripheral of the fiber core. The beam deflection unit 1506 includes a chamfered end surface. The chamfered end surface is covered by a metal coating layer. In some embodiments, the beam deflection unit 1506 may be similar to the LITT lateral ablation probe 700 as described in FIG. 7. Specifically, the beam deflection unit 1506 includes a main body, a connection surface and a coating layer. The main body includes the fiber core and the hard cladding structure located at an outer peripheral of the fiber core. The fiber core is made of pure silicon oxide material. The hard cladding structure is made of TECS material. An end of the main body has an angle of inclination, which is the chamfered end surface. At this time, the end surface of the main body and an axis of the main body form a certain angle, which ranges from 0 to 90°, excluding the two terminal points of the range. The connection surface is a joint interface between the main body and the coating layer. The connection surface is formed by brightening and cleaning the chamfered end surface. The connection surface is coated with the coating layer (e.g., a noble metal coating layer). The noble metal may include, for example, gold, silver, and metal of the platinum family. The coating layer is formed performing, e.g., magnetron sputtering coating on the connection surface. In some embodiments, an angle between the coating layer and the axis of the main body 710 is, for example, 35°, 37°, 39°, 41°, 43°, 45°, 47°, 49°, etc.

In some embodiments, a truncated axial cylinder of the beam deflection unit 1506 has a certain length. The truncated axial cylinder is a cylinder between a surface of a beam incident end of the beam deflection unit 1506 and a chamfered point of the chamfered end surface. The chamfered point is a point on the chamfered end surface having a nearest distant to the surface of the beam incident end of the beam deflection unit 1506. A length of the truncated axial cylinder is, for example, 2 μm, 5 μm, 8 μm, 10 μm, etc. In some embodiments, the length of the truncated axial cylinder is 5 μm. At this time, the spectral deflection effect of the beam deflection unit 1506 is preferable.

The spring torsion coil 1508 is configured at a front end of the OCT probe 1500. Illustratively, an end of the spring torsion coil 1508 is abut against the start terminal of the second lens 1504. The spring torsion coil 1508 provides a rotary torque support for the OCT probe 1500 through its stretchable, compressible, and torsional properties.

The optical sleeve 1510 is used to accommodate the spring torsion coil 1508, the first lens 1502, the second lens 1504, and the beam deflection unit 1506. In some embodiments, the optical sleeve 1510 is a tubular optical element, such as a tubular optical glass. The spring torsion coil 1508, the first lens 1502, the second lens 1504 and the beam deflection unit 1506 are configured in the optical sleeve 1510 in sequence. At the same time, the optical sleeve 1510 has a relatively high optical transmissivity. The light beam deflected by the beam deflection unit 1506 exits the OCT probe 1500 through the optical sleeve 1510.

The filler 1512 is filled inside the optical sleeve 1510. In some embodiments, the filler 1512 is an optical adhesive. A curing reaction occurs in a period of time after the filler 1512 is filled into the optical sleeve 1510. By injecting the filler 1512 into the optical sleeve 1510, the filler 1512 may be filled in all gaps among the first lens 1502, the second lens 1504, the beam deflection unit 1506, and the optical sleeve 1510, such that the first lens 1502, the second lens 1504, and the beam deflection unit 1506 may be fixed relative to the optical sleeve 1510. In some embodiments, a refractive index of the filler 1512 is far less than that of the second lens (e.g., the micro plano-convex cylindrical lens), thereby avoiding the effect on the spreading of the light beam in the OCT probe 1500.

In some embodiments, ignoring the influence of the length of the truncated axial cylinder of the beam deflection unit 1506, the focal length of the OCT probe 1500 is assumed to be z₀, a diameter of the focal spot is assumed to be 2ω₀, any length for the light spectrum exiting the OCT probe 1500 is denoted as z, a diameter of a light spot corresponding to the length z is denoted as 2ω. The ABCD optical Gaussian transmission matrix T of the OCT probe 1500 may be expressed as:

$$T = [ \begin{matrix}1 & 0 \\ \frac{1 - n}{r} & n\end{matrix} ] [ \begin{matrix}1 & L \\ 0 & 1\end{matrix} ] [ \begin{matrix}1 & 0 \\ 0 & \frac{1}{n}\end{matrix} ] = [ \begin{matrix}1 & L \frac{1}{n} \\ \frac{1 - n}{r} & [ \frac{1 - n}{r} L + n ] \frac{1}{n}\end{matrix} ] ,$$

The Gauss transmission moment M corresponding to the any length z for the light spectrum exiting the OCT probe 1500 is expressed as:

$$M = [ \begin{matrix}A & B \\ C & D\end{matrix} ] = [ \begin{matrix}1 & z \\ 0 & 1\end{matrix} ] T [ \begin{matrix}1 & b \\ 0 & 1\end{matrix} ] ,$$

In the meanwhile, the central wave number of the light source is defined as k₀, a given wavelength value within a bandwidth of the light spectrum is λ, and the focal length z₀ of the OCT probe for b of different values may be determined as:

$${z}_{0} ( b ) = \frac{\frac{1}{Re ( \frac{1}{\frac{{Aq}_{0} + B}{{Cq}_{0} + D}} )}}{1 + {[ \frac{\frac{1}{Re ( \frac{1}{\frac{A \frac{π {k}_{0}}{λ} i + B}{C \frac{π {k}_{0}}{λ} i + D}} )} λ}{{( \frac{1}{\sqrt{- \frac{\frac{π lm ( \frac{1}{A \frac{π {k}_{0}}{λ} i + B} )}{C \frac{π {k}_{0}}{λ} i + D}}{λ}}} )}^{2} π} ]}^{2} λ}$$

The radius of the focal spot ω₀ for b of different values may be determined as:

$${ω}_{0} ( b ) = \sqrt{\frac{{( \frac{1}{\sqrt{- \frac{\frac{π lm ( \frac{1}{A \frac{π {k}_{0}}{λ} i + B} )}{C \frac{π {k}_{0}}{λ} i + D}}{λ}}} )}^{2}}{1 + {[ \frac{{( \frac{1}{\sqrt{- \frac{\frac{π lm ( \frac{1}{A \frac{π {k}_{0}}{λ} i + B} )}{C \frac{π {k}_{0}}{λ} i + D}}{λ}}} )}^{2} π}{\frac{1}{Re ( \frac{1}{\frac{A \frac{π {k}_{0}}{λ} i + B}{C \frac{π {k}_{0}}{λ} i + D}} )} λ} ]}^{2}}}$$

It can be seen that the focal length and the focal spot size of the OCT probe 1500 may vary by adjusting the length of the first lens 1502 (e.g., the coreless lens), thereby changing the working distance of the OCT probe 1500, such that the OCT probe may be applicable for pathological imaging of tissue of large size.

FIG. 16 is a schematic diagram illustrating manufacturing of an FBG sensor according to some embodiments of the present disclosure.

In this embodiment, the FBG sensor is obtained by irradiating a specific raw material for manufacturing the FBG sensor using a FBG irradiating device 1600. The specific raw material needs to satisfy specific parameter conditions, which are set forth in the above descriptions, and are not repeated here. The FBG irradiating device 1600 includes a terminal 1601, a laser device controller 1602, a laser device 1603, beam correction devices 1604-1 and 1604-2, a slit diaphragm 1605, an ultraviolet coated lens 1606, a phase mask 1607, a sliding rail 1609, a fixing device 1610, a motion driver 1611, and a motion driver controller 1612.

The terminal 1601 is connected to and controls the laser device controller 1602 and the motion driver controller 1612. The terminal 1601 may be, for example, a computer. The laser device controller 1602 is connected to the laser device 1603 and controls the laser device 1603 to emit laser. The laser device 1603 may be an excimer pulsed laser device of a characteristic wavelength (e.g., 248 nm). As for the laser device 1603, a power of the laser generated by the laser device 1603 may be, for example, 8 W, 10 W, 12 W, 15 W, etc.; a pulse frequency of the laser device 1603 may be, for example, 90 Hz, 100 Hz, 120 Hz, etc.; pulse energy of the laser device 1603 may be, for example, 100 mJ, 120 mJ, 140 mJ, etc.

Correspondingly, the laser generated by the laser device 1603 is a flat-topped laser beam with an almost uniform flux (energy density). The light spot of the laser beam is characterized by a rectangular flat-top. A central wavelength of the laser beam is, e.g., 248 nm. A pulse duration of the laser beam is, e.g., 10 ns, 12 ns, 15 ns, 20 ns, etc.; the rectangular flat-top is, e.g., 4×1 mm², 6×1.5 mm², 10×3 mm²; a divergence angle of the laser beam is, e.g., 2×1 mrad², 3×2 mrad², 4×2 mrad², etc.

The beam correction devices 1604-1 and 1604-2 are used to deflect the laser beam and correct a light path of the laser beam. In this embodiment, the beam correction devices 1604-1 and 1604-2 are excimer laser 45° reflecting mirrors with a characteristic wavelength of 248 nm, each of which is used to deflect the laser beam over an angle of 45° and correct the light path of the laser beam.

The slit diaphragm 1605 is used to restrict the propagation of the laser beams in a specific direction. A size of the slit diaphragm 1605 (i.e., a width of a slit of the slit diaphragm 1605) is, for example, 2-6 mm. In some embodiments, the size of the slit diaphragm 1605 is 2 mm. In some embodiments, the size of the slit diaphragm 1605 is 3 mm. In some embodiments, the size of the slit diaphragm 1605 is 4.5 mm. In some embodiments, the size of the slit diaphragm 1605 is 6 mm.

The ultraviolet coated lens 1606 is used to focus the laser beam in the ultraviolet domain. In this embodiment, the ultraviolet coated lens 1606 may be an ultraviolet coated fused quartz plano-convex cylindrical lens. The ultraviolet coated fused quartz plano-convex cylindrical lens may focus (with a focal length of, e.g., 150 mm, 200 mm, 250 mm, etc.) the laser beam to the phase mask 1607. A characteristic wavelength of the ultraviolet coated fused quartz plano-convex cylindrical lens is 100-600 nm. In some embodiments, the characteristic wavelength of the ultraviolet coated fused quartz plano-convex cylindrical lens is 200-500 nm. In some embodiments, the characteristic wavelength of the ultraviolet coated fused quartz plano-convex cylindrical lens is 245-440 nm.

The phase mask 1607 is configured in front of the specific raw material 1608. A striped light spot may be formed on the raw material 1608 by irradiating the phase mask 1607 using the laser beam. In this embodiment, the phase mask 1607 is an ultra-bandwidth (for example, 1460-1600 nm) phase mask of ultraviolet radiation with a 248 nm characteristic wavelength. A width of the striped light spot is, for example, 10 mm, 15 mm, 20 mm, 25 mm, etc.; A height of the striped light spot is, for example, 28 μm, 32.4 μm, 40 μm, 50 μm, etc.

The motion driver controller 1612 is connected to and controls the motion driver 1611 to drive the fixing device 1610 to move, e.g., translate and/or rotate.

A process for manufacturing the FBG sensor by irradiating the specific raw material using the FBG irradiating device 160 includes one or more of the following steps.

S16-1: two ends of the specific raw material 1608 are fixed on the sliding rail 1609 and the fixing device 1610, respectively. The fixing device 1610 is fixedly connected to the motion driver 1611. The fixing device 1610 can be a clamp. The clamp may fixedly grip one end of the specific raw material 1608.

S16-2: The laser device 1603 is controlled, by the laser device controller 1602, to emit laser. The emitted laser passes through the beam correction devices 1604-1 and 1604-2, the slit diaphragm 1605, the ultraviolet coated lens 1606, and the phase mask 1607 sequentially, and forms the striped light spot on the surface of the specific raw material 1608.

S16-3: the motion driver 1611 is controlled, by the motion driver controller 1612, to drive the specific raw material 1608 to move. When the specific raw material 1608 moves, the FBG sensor may be formed by irradiating the specific raw material 1608 using the laser.

The fixing device 1610 is fixedly connected to the motion driver 1611. When the motion driver controller 1612 controls the motion driver 1611 to drive the fixing device 1610 to move, the specific raw material 1608 also moves (translate, rotate, etc.) along with the fixing device 1610. During the motion of the specific raw material 1608, the laser beam irradiates the surface of the specific raw material 1608 in the striped light spot, so that the refractive index of the fiber core is modulated periodically. The periodic core index modulations generate core modes. These core modes reflect or transmit through index boundaries and interfere with each other. In turn, input light is strongly reflected merely at a specific wavelength determined by a certain phase matching condition. The specific wavelength is referred to as a Bragg wavelength of FBG. The phase matching condition is also referred to as a Bragg condition. The FBG sensor is formed accordingly.

Benefits brought about according to the embodiments of the present disclosure may include but not limited to: (1) by using the thermocouple and/or the FBG sensor in temperature measurement, which is more accurate than MRTI, at the far end of the LITT probe, a real time and efficient feedback of the temperature measurement of the LITT may be realized. Thus, damages on tissue caused by excessive temperature of the far end of the LITT probe may be avoided, and postoperative sequelae may be reduced; (2) by applying of the preparation technologies for carving the cone-shaped surface and the arabesquitic pattern on the LITT ablation probe, the LITT ablation applicator may be manufactured. The LITT ablation applicator may have a more uniform spatial distribution of the vectorial optical energy than that of an LITT ablation applicator manufactured using a conventional local speckle carving technology. In combination with the temperature measurement mechanism of the temperature measurement element as set forth above, ablation of surrounding tissue becomes more uniform and radical. In the meanwhile, combining with thermal characteristics of tissue and heat injury algorithms based on the LITT circumferential ablation probe 900, both a protection of healthy tissue in the treatment and ablation effect of ablation pathology may have remarkable improvements; (3) by adopting the OCT pathological imaging mechanism, the OCT device may detect remaining cancerous pathology of the ablated lesion in real time, and implement imaging of the cancerous pathology remained at an edge of the ablated lesion, such that a fast supplementary ablation may be performed, and the tumor residue may be reduced and a recurrence probability may be minimized; (4) by providing the driving motor compatible with MRI, two degrees of freedom of the LITT probe motion mechanism is introduced, thereby realizing a translational motion freedom and a rotational motion freedom at high piezoelectric sensing precision. The translation control mechanism and the rotation control mechanism configured on the interface platform are driven to move by controlling the translational motion freedom cable (translation cable) and the rotational motion freedom cable (rotation cable), thereby achieving accurate positioning of the MRg-LITT ablation, with finely controllable motion, ensuring an accurate ablation treatment and a perfect compatibility with the LITT lateral and circumferential ablation probes, and having a huge clinical value.

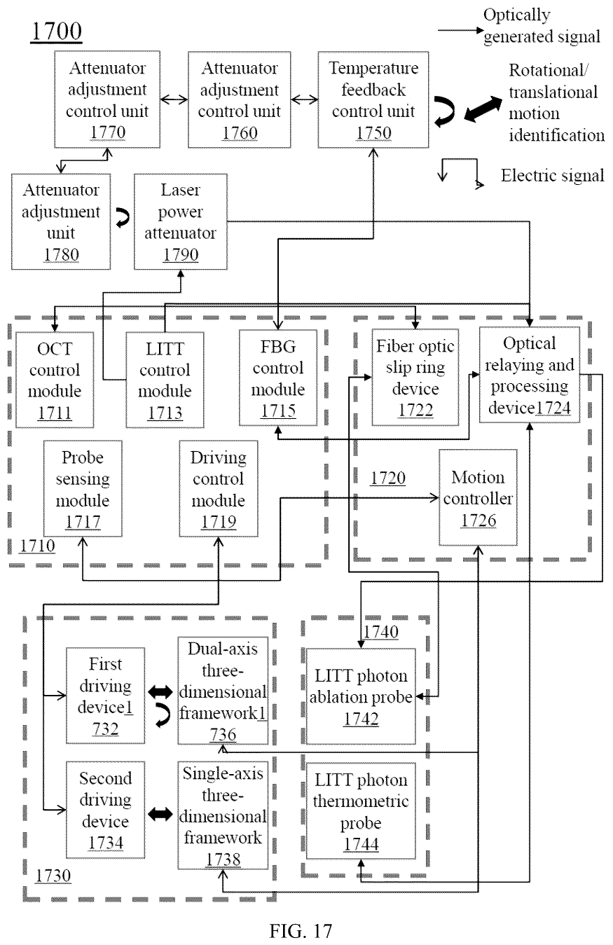

FIG. 17 is a schematic diagram of a medical treatment apparatus according to some embodiments of the present disclosure.

In some embodiments, the medical treatment apparatus 1700 may be an apparatus that integrates the various devices and components (local or remote) in the medical treatment system 100. As illustrated in the figure, the medical treatment apparatus 1700 may include an MRI device (not shown in the figure), a LITT device (not shown in the figure), a temperature measurement device (not shown in the figure), an OCT device (not shown in the figure), a control device 1710, an interface platform hub control module 1720, a driving assembly 1730, a probe set 1740, a temperature feedback control unit 1750, a laser dose control unit 1760, an attenuator adjustment control unit 1770, an attenuator adjustment unit 1780, and a laser power attenuator 1790.

The MRI device may be used to generate one or more MRI images by imaging a specific region including the target object (e.g., a cancer lesion). The one or more MRI images may be real time MRI images or nonreal-time MRI images. The one or more MRI images may include a three-dimensional image or multiple two-dimensional images (e.g., a transverse image, a coronal image, and a sagittal image), and characterize information (such as a position, a size, etc.) of the target object in the three-dimensional space. The information of the target object in the three-dimensional space provided by the one or more MRI images may be used for planning, before a treatment for a patient, a route along which an LITT probe, an OCT probe, and/or a temperature measurement element of the probe set 1740 arrive a position where the target object locates through body tissue of the patient (also referred to as probe planning). The information of the target object in the three-dimensional space provided by the one or more MRI images may also be used for guiding the LITT probe, the OCT probe, and/or the temperature measurement element to access the body tissue of the patient according to the planned route, and arrive the position where the target object locates (referred to as probe guidance). In some embodiments, the one or more MRI images may be displayed on a terminal (e.g., the terminal 150). The probe planning and the probe guidance may be implemented using the terminal (e.g., physical elements such as a touch screen, a mouse, a keyboard, etc., of the terminal 150) by a user, such as a doctor based on the one or more MRI images. In some embodiments, the probe planning and the probe guidance may also be implemented by the system automatically based on the one or more MRI images.

In some embodiments, the MRI device may also perform MRTI on the specific region including the target object, and generate one or more thermal images. The one or more thermal images may be registered with the one or more MRI images. Anatomical structure information of the specific region including the target object and temperature variation information of corresponding positions may be characterized by the registered thermal images and MRI images.

The LITT device may be used to emit laser and treat the target object using thermal effect of the laser. The LITT device includes a laser device, an LITT probes (e.g., an LITT lateral ablation probe, an LITT circumferential ablation probe), and channels (such as fibers) and interfaces that connect the laser device and the LITT probe. In some embodiments, the laser device may include a tunable laser diode and/or an untunable laser diode. A power of the tunable laser diode is adjustable within a specific range, such as 0-500 W, 0-250 W, 0-50 W, 0-10 W, 1-8 W, etc. A power of the untunable laser diode is a specific value, for example, 1 W, 3 W, 5 W, 8 W, 10 W, 12 W, 15 W, 20 W, 30 W, 60 W, 100 W, etc. The laser has a specific characteristic wavelength, for example, 840 nm, 980 nm, 1064 nm, 1300 nm, etc.

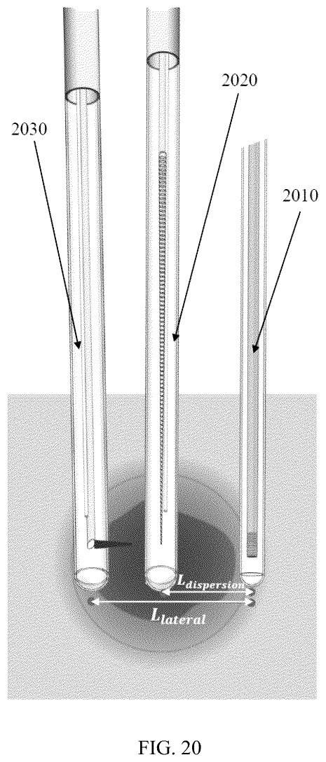

The temperature measurement device includes a temperature measurement element. The temperature measurement element may be used to measure temperature(s) of the target object or a specific position on its edge (e.g., a position on the edge of the target object farthest from the LITT probe) to determine tissue temperature(s) of the entire target object so as to ensure therapeutic effect. A distance between the LITT probe and the temperature measurement element is smaller than the size of the target object. In some embodiments, the edge of the target object may have an irregular shape. The edge of the target object may be deemed as an equivalent circle. For example, a minimum circumscribed circle of the target object may be determined. The minimum circumscribed circle may be designated as the equivalent circle of the target object. The size of the target object may be, for example, a diameter of the equivalent circle. The setting of the position of the temperature measurement element and the LITT probe can be referred to other figures (e.g., FIG. 20) and the descriptions thereof in the present disclosure, which are not repeated here.

The temperature measurement element may include a thermocouple (such as a K-type thermocouple), an LITT photon thermometric probe, etc. In this embodiment, the temperature measurement element may be an LITT photon thermometric probe. The LITT photon thermometric probe may include an FBG thermometric probe. The FBG thermometric probe includes an optical fiber made of specific raw material. The specific raw material may be exposed to ultraviolet light in a certain wavelength range (e.g., 240-244 nm, 244-248 nm, 248-252 nm, 252-256 nm, etc.), so that the refractive index of a fiber core of the optical fiber is modulated periodically. The periodic core index modulations generate core modes. These core modes reflect or transmit through index boundaries and interfere with each other. In turn, input light is strongly reflected merely at a specific wavelength determined by a certain phase matching condition. The specific wavelength is referred to as a Bragg wavelength of FBG. The phase matching condition is also referred to as a Bragg condition. The FBG thermometric probe is formed accordingly. The FBG thermometric probe may be used for monitoring temperatures of interstitial tissue at specific positions in real time during a dispersive irradiation of laser ablation.

The OCT device may detect reflection signals, scattering signals, etc., of biological tissue based on light transmittance of biological structures, and convert the detected signals into electric signals to generate one or more OCT images. The one or more OCT images may be real time OCT images, or non-real time OCT images. The OCT device includes a light source, an OCT probe, an interference component, and optical fibers and interfaces that connect the various components of the OCT device. The light source uses a low-coherence light source to improve a vertical resolution of the OCT imaging. In this embodiment, the OCT device may be dual-mode OCT, and the light source may generate two light signals of different parameters (e.g., a bandwidth and a central wavelength). Merely by way of example, the dual-mode OCT generates a first light signal of a bandwidth exceeding 160 nm and a central wavelength at 840 nm, and a second light signal of a swept-frequency range exceeding 100 nm and a central wavelength at 1300 nm. The dual-mode OCT may facilitate pathological imaging with approximating 1 μm resolution and centimeter-level depth so as to implement real-time pathological imaging of ablating pathology and actual morphology of apoptosis, and further facilitate a pathological evaluation.

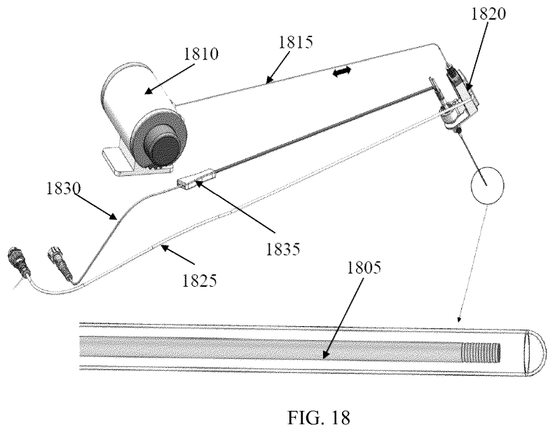

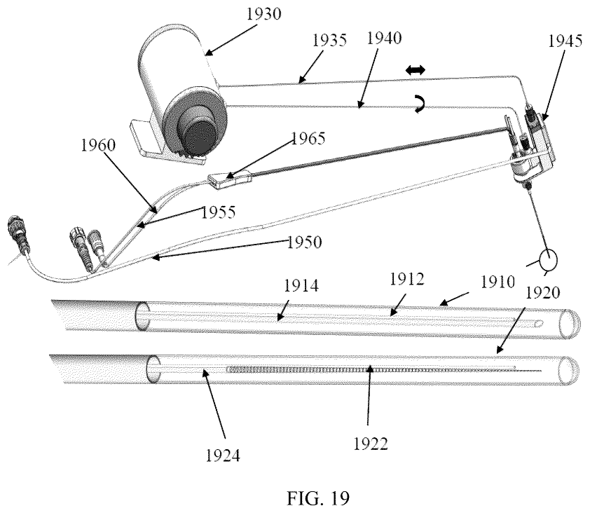

In some embodiments, the probe set 1740 includes an LITT photon ablation probe 1742 of the LITT device and the LITT photon thermometric probe 1744 of the temperature measurement device. In some embodiments, the above-mentioned LITT probe (e.g., the LITT lateral ablation probe, the LITT circumferential ablation probe) and the OCT probe may be integrated as an integrated probe that integrating medical diagnosis and treatment (hereinafter referred to as integrated probe for short). The LITT photon ablation probe 1742 may be an integrated probe. The LITT photon thermometric probe 1744 and the LITT photon ablation probe 1742 are independent of each other (e.g., having independent structures and being controlled independently). In this embodiment, the LITT photon ablation probe 1742 and the LITT photon thermometric probe 1744 of the temperature measurement device are arranged at different positions relative to the target object. The structures and position setting of the LITT photon ablation probe 1742 and the LITT photon thermometric probe 1744 can be referred to other figures (e.g., FIGS. 18-20) and the descriptions thereof in this disclosure, which are not repeated here.

The control device 1710 may be used to control one or more devices or components of the medical treatment apparatus 1700 so as to perform corresponding operations. The control device 1710 may generate corresponding instructions based on controlled devices or components and operations to be performed. The instructions are transmitted to the controlled devices or components in the form of electric signals, so that the controlled devices or components perform the corresponding operations. The control device 1710 may include, for example, a microcontroller (MCU), a central processor (CPU), a programmable logic device (PLD), an application specific integrated circuit (ASIC), a single-chip microcomputer (SCM), a system on chip (SOC), etc. In some embodiments, the control device 1710 may be an industrial personal computer.

In some embodiments, the control device 1710 integrates an OCT control module 1711, an LITT control module 1713, an FBG control module 1715, a probe sensing module 1717, and a driving control module 1719. The OCT control module 1711 is configured to control the OCT imaging of the target object. The OCT control module 1711 may generate instructions to control the OCT device to emit and receive optical signals and/or set imaging parameters (e.g., an optical signal bandwidth, a central wavelength of the optical signal, imaging time, image contrast, etc.). For example, the OCT control module 1711 may control the OCT device to emit a dual-mode OCT optical signal including a first optical signal of a bandwidth of 160 nm and a central wavelength at 840 nm, and a second optical signal of a swept-frequency range exceeding 100 nm and a central wavelength at 1300 nm. The dual-mode OCT optical signal is transmitted to a fiber optic slip ring device 1722, where motion control and spatial coupling processing are performed, and is transmitted to the OCT probe finally. In some embodiments, when the LITT photon ablation probe 1742 ablates the target object, the OCT control module 1711 may also receive pathology diagnostic signals (e.g., optical signals carrying pathological features of tissue) to generate one or more OCT images.

The LITT control module 1713 may control the LITT photon ablation probe 1742 to perform an ablation treatment on the target object. In some embodiments, a laser device controller is integrated into the LITT control module 1713. The laser device controller may control the laser device of the LITT device (e.g., the tunable laser diode and/or the non-tunable laser diode) to emit laser to provide an ablation energy source for the entire LITT. The LITT control module 1713 may control the laser device to emit laser, and the emitted laser is transmitted to the LITT photon ablation probe 1742 through an optical relaying and processing device 1724.

Illustratively, the LITT control module 1713 controls the laser device of the LITT device to emit laser of a specific wavelength (e.g., 980 nm or 1064 nm) at a specific power (e.g., at a power selected in a range of 1-8 W). The laser is transmitted into the optical relaying and processing device 1724 for optical relayed physical connection, and further transmitted to the LITT photon ablation probe 1742. Then the LITT photon ablation probe 1742 emits an ablation laser to ablate target tumor tissue.

The FBG control module 1715 may control the temperature measurement element to obtain a temperature of a specific position at the edge of the target object. A temperature measurement signal of the temperature measurement element (such as a heat source optical signal of the LITT photon thermometric probe 1744) may be transmitted through the optical relaying and processing device 1724. In some embodiments, the FBG control module 1715 may control the LITT photon thermometric probe 1744 to detect a temperature of a specific location at the edge of the target object (e.g., a farthest edge of tumor tissue, and the farthest edge is determined by setting the LITT photon ablation probe 1742 as a geometric distance measuring point ablated with LITT photons) in real time. The temperature is transmitted to the optical relaying and processing device 1724, and further transmitted to the FBG control module 1715. The optical relaying and processing device 1724 may support and ensure annular outputs of optical signals, so as to avoid an optical path conflict between an optical path for thermometric output carrying temperature information and an optical path for thermometric input. The FBG control module 1715 may modulate a targeted spectral signal using a carried temperature scale, and convert the spectral signal into a specific temperature in the form of an electric signal.

The probe sensing module 1717 may detect real-time position(s) of the LITT photon ablation probe 1742 and/or the LITT photon thermometric probe 1744 in real time by controlling a motion controller 1726. For example, the probe sensing module 1717 may trigger traversing of feedback variation of a position signal of the motion controller 1726 by generating an electric signal. The motion controller 1726 may monitor and interact with a dual-axis three-dimensional framework 1736 and a single-axis three-dimensional framework 1738 in real time, e.g., through motion sensors so as to obtain position information of the LITT photon ablation probe 1742 and the LITT photon thermometric probe 1744 within a specific part of the patient (e.g., the brain or other organs of the human body) in real time. The probe sensing module 1717 may also send control signals to the motion controller 1726 in real time to issue motion triggering instructions and receive position feedback processing signals. The motion triggering instructions are used to cause the motion controller 1726 to control the LITT photon ablation probe 1742 and/or the LITT photon thermometric probe 1744 to move. The position feedback processing signals are used to estimate and determine subsequent motions (e.g., moving on, reorientating, retreating, etc.) of the LITT photon ablation probe 1742 and/or the LITT photon thermometric probe 1744 based on real-time position(s) of the LITT photon ablation probe 1742 and/or the LITT photon thermometric probe 1744.

In some embodiments, at least one motion sensor is configured on each of the dual-axis three-dimensional framework 1736 and the single-axis three-dimensional framework 1738, respectively. The at least one motion sensor set on the dual axis three-dimensional framework 1736 is used to monitor the motion of the LITT photon ablation probe 1742. The at least one motion sensor set on the single-axis three-dimensional framework 1738 is used to monitor the motion of the LITT photon thermometric probe 1744. The motion sensor may be, e.g., a piezoelectric sensor, an inductive sensor, an eddy current sensor, or the like.

The driving control module 1719 may control a first driving device 1732 and/or a second driving device 1734 of the driving assembly 1730 to drive the LITT photon ablation probe 1742 and/or the LITT photon thermometric probe 1744 to move by sending instruction through electric signals. The first driving device 1732 is also referred to as ablation end driving device, which is connected to the LITT photon ablation probe 1742 and drives the LITT photon ablation probe 1742 to move (e.g., translate, rotate), thereby realizing an accurate motion control of two degrees of freedom of the LITT photon ablation probe 1742. The second driving device 1734 is also referred to as a thermometric end driving device, which is connected to the LITT photon thermometric probe 1744 and drives the LITT photon thermometric probe 1744 to move (e.g., translate), thereby realizing an accurate motion control of one degree of freedom of the LITT photon thermometric probe 1744. The implementation of the motions of the LITT photon ablation probe 1742 and the LITT photon thermometric probe 1744 need mechanical clamping and support for power transmission provided by the dual-axis three-dimensional framework 1736 and the single-axis three-dimensional framework 1738, respectively. The dual-axis provides two power transmission of two different degrees of freedom, and the single-axis provides a power transmission of a single degree of freedom.

The interface platform hub control module 1720 integrates middle section control or relay control of one or more components or elements of the medical treatment apparatus 1700. Merely for illustration, the interface platform hub control module 1720 integrates the above-mentioned fiber optic slip ring device 1722, the optical relaying and processing device 1724, and the motion controller 1726. In some embodiments, the interface platform hub control module 1720 includes a packaging box. The fiber optic slip ring device 1722, the optical relaying and processing device 1724, and the motion controller 1726 are arranged in the packaging box and are connected to corresponding components or elements through corresponding channels. For example, the fiber optic slip ring device 1722 is connected to the OCT probe through an OCT probe channel (optical fiber); the optical relaying and processing device 1724 is connected to the LITT probe and the LITT photon thermometric probe 1744 through an LITT ablation probe channel and an LITT thermometric probe channel, respectively; the motion controller 1726 is connected to the motion sensor through a channel accommodating a cable of the motion sensor.

The fiber optic slip ring device 1722 may be disposed on a transmission path (e.g., at a rotating joint) of the optical signal generated by the OCT device. The fiber optic slip ring device is used to ensure continuous transmission of the optical signals. For the dual-mode OCT, a multi-channel fiber optic slip ring device (such as a dual-channel fiber optic slip ring device that adapts the two different central wavelengths of the optical signals) also referred to as multi-mode fiber optic slip ring, may be used.

The optical relaying and processing device 1724 is used to process the optical signals and ensure continuous transmission of the optical signals. In some embodiments, the laser emitted by the laser device of the LITT device is processed by the optical relaying and processing device 1724 and transmitted to the LITT probe. The optical relaying and processing device 1724 may adjust at least one parameter (e.g., power, frequency, etc.) of the laser. For example, the optical relaying and processing device 1724 may compensate an attenuation of the laser such that the power of the laser reaches a specific power, or perform an attenuation processing on the laser such that the power of the laser is reduced to meet the requirement of the medical treatment. In some embodiments, the optical relaying and processing device 1724 supports and guarantees annular outputs of optical signals of the temperature measurement device, so as to avoid an optical path conflict between an optical path for thermometric output carrying temperature information and an optical path for thermometric input, which may cause interruptions or mutual interferences of optical signals.

The motion controller 1726 may control the movement of the LITT photon ablation probe 1742 and/or the LITT photon thermometric probe 1744 based on instructions of the probe sensing module 1717. During a treatment of the target object, the motion controller 1726 may control the LITT photon ablation probe 1742 to pass through the LITT ablation probe channel configured on the dual-axis three-dimensional framework 1736, traverse a specific part of the patient (e.g., traverse the skull of the patient, access the brain of the patient), and arrive at a position where the target object locates according to the probe planning and probe guidance of the LITT photon ablation probe 1742. In the meanwhile, the motion controller 1726 may control the LITT photon thermometric probe 1744 to pass through the LITT thermometric probe channel configured on the single-axis three-dimensional framework 1738, traverse a specific part of the patient (e.g., traverse the skull of the patient, access the brain of the patient), and arrive at a position where the target object locates according to the probe planning and probe guidance of the LITT photon thermometric probe 1744. Then temperature(s) of an edge or surrounding tissue of the target object at a specific distance from the LITT photon thermometric probe 1744 may be measured. The dual-axis three-dimensional framework 1736 and the single-axis three-dimensional framework 1738 are configured at nearby positions of a specific part (e.g., the head, the chest, a limb, etc.) of the patient.

The driving assembly 1730 is used to drive the LITT photon ablation probe 1742 and/or the LITT photon thermometric probe 1744 to move, so that the LITT photon ablation probe 1742 and/or the LITT photon thermometric probe 1744 may reach or move away from a specific position (e.g., where the target object is located). The motion of the LITT photon ablation probe 1742 may include a translational motion and a rotational motion. The motion of the LITT photon thermometric probe 1744 may include a translational motion. In some embodiments, the driving assembly 1730 includes a first driving device 1732 (also referred to as ablation end driving device) and a second driving device 1734 (also referred to as thermometric end driving device). The first driving device 1732 and the second driving device 1734 may control the motion of the LITT photon ablation probe 1742 and The LITT photon thermometric probe 1744, respectively. The first driving device 1732 is independent of the second driving device 1734. Each of the first driving device 1732 and the second driving device 1734 may include at least one driving motor, at least one cable, and at least one motion control mechanism. The LITT photon ablation probe 1742 and the LITT photon thermometric probe 1744 are physically connected to corresponding motion control mechanisms through respective cables. Through the corresponding cables and motion control mechanisms, forces output by the corresponding driving motors may be transmitted to the LITT photon ablation probe 1742 and the LITT photon thermometric probe 1744 so as to control their motions.

In this embodiment, the first driving device 1732 is coupled to the LITT photon ablation probe 1742, and controls the translational motion and rotational motion of the LITT photon ablation probe 1742. The first driving device 234 includes a first driving motor, a first translation cable, a first translation control mechanism, a first rotation cable, and a first rotation control mechanism. The first translation control mechanism is used to control the translational motion of the LITT photon ablation probe 1742. The first rotation control mechanism is used to control the rotational motion of the LITT photon ablation probe 1742. The first translation cable and the first rotation cable are connected to the first translation control mechanism and the first rotation control mechanism, respectively. The first translation control mechanism and the first rotation control mechanism are connected to the LITT photon ablation probe 1742, and control the translational motion and the rotational motion of the LITT photon ablation probe 1742 through the first translation cable and the first rotation cable, respectively. The first driving motor may drive the first translation cable and/or the first rotation cable to move according to a requirement, so as to control the translational motion and/or rotation motion of the LITT photon ablation probe 1742, thereby realizing an accurate motion control of two degrees of freedom of the LITT photon ablation probe 1742. In some embodiments, the first translation control mechanism and the first rotation control mechanism may be configured on the dual-axis three-dimensional framework 1736.

The second driving device 1734 is coupled to the LITT photon thermometric probe 1744, and controls the translational motion of the LITT photon thermometric probe 1744. The second drive device 1734 includes a second driving motor, a second translation cable, and a second translation control mechanism. The second translation control mechanism is used to control the translational motion of the LITT photon thermometric probe 1744. The second translation cable is connected to the second translation control mechanism. The second translation control mechanism is connected to the LITT photon thermometric probe 1744, and controls the translational motion of the LITT photon thermometric probe 1744 through the second translation cable. The second driving motor may drive the second translation cable to move according to a requirement, so as to control the translational motion of the LITT photon thermometric probe 1744, and thereby realizing an accurate motion control of one degree of freedom of the LITT photon thermometric probe 1744. In some embodiments, the second translation control mechanism may be configured on the single-axis three-dimensional framework 1738.

In some embodiments, each of the cables mentioned above (e.g., the first translation cable, the first rotation cable, the second translation cable) may be a specially made wire. The specially made wire has a relatively high stiffness and a relatively low elastic modulus, and is capable of transmitting a torque in a transmitting ratio of 1:1 in real time, thereby ensuring a motion accuracy of the LITT photon ablation probe 1742 and/or the LITT photon thermometric probe 1744.

The dual-axis three-dimensional framework 1736 and the single-axis three-dimensional framework 1738 may carry one or more components or elements of the medical treatment apparatus 1700. Exemplarily, the components or elements carried on the dual-axis three-dimensional framework 1736 include the LITT ablation probe channel of the LITT photon ablation probe 1742, at least one motion sensor, the first motion control mechanism (such as the first translation control mechanism, the first rotation control mechanism), etc. The components or elements carried on the single-axis three-dimensional framework 1738 include the LITT thermometric probe channel of the LITT photon thermometric probe 1744, at least one motion sensor, the second translation control mechanism, etc. The dual-axis three-dimensional framework 1736 and the single-axis three-dimensional framework 1738 may be fixed to a specific part of the patient (e.g., two different positions of the skull), and form a stable connection with the specific part of the patient to avoid a relative displacement. The components or elements carried on the dual-axis three-dimensional framework 1736 and the single-axis three-dimensional framework 1738 may be fixed to the dual-axis three-dimensional framework 1736 and the single-axis three-dimensional framework 1738.

The temperature feedback control unit 1750 is used to monitor the temperature of a specific position at the edge of the target object measured by the LITT photon thermometric probe 1744, and ensure that the measured temperature is within a preset temperature range. The preset temperature range is set by the user, according to default settings of the system, etc. For example, the preset temperature range is 44±0.5° C., 44±1° C., 45±0.5° C., 45±1° C., 46±0.5° C., 46±1° C., 47±0.5° C., 47±1° C., 48±0.5° C., 48±1° C., etc. In some embodiments, the preset temperature range is 46±1° C. A better therapeutic effect may be achieved when a temperature of tissue of the target subject is in the preset temperature range. When the medical treatment apparatus 1700 works in such a condition, the treatment effect can be relatively better, and the treatment success rate and treatment efficiency can be relatively high. The temperature feedback control unit 1750 is connected to the FBG control module 1715. The FBG control module 1715 may transmit the temperature measured by the LITT photon thermometric probe 1744 to the temperature feedback control unit 1750 in the form of an electric signal. The transmission of the measured temperature may be in real time or at intervals (e.g., periodically). In some embodiments, when the temperature measured by the LITT photon thermometric probe 1744 exceeds the preset temperature range, the temperature feedback control unit 1750 may determine a difference (i.e., temperature difference) between the measured temperature and the preset temperature range, and output the difference to the laser dose control unit 1760 to adjust a dose of the laser output by the LITT photon ablation probe 1742, so that the temperature measured by the LITT photon thermometric probe 1744 may return back to the preset temperature range.

The laser dose control unit 1760 may determine a target output dose value (i.e., a target value of the dose to be output) of the laser. The laser dose control unit 1760 may be connected to the temperature feedback control unit 1750. The laser dose control unit 1760 may obtain the temperature difference determined by the temperature feedback control unit 1750, and determine the target output dose value based on the temperature difference. The target output dose value may be a total dose value of the laser in a certain period of time, or a dose value of the laser at each moment. A variation trend of the temperature of the tissue at the position where the LITT photon thermometric probe 1744 is located may be determined based on the total dose value of the laser in the certain period of time or the dose value of the laser at each moment, so that the temperature measured by the LITT photon thermometric probe 1744 may return back to the preset temperature range.

In some embodiments, the above-described process for determining the variation trend may be determined based on a model or a specific algorithm. The model may be, for example, a machine learning model. Exemplary machine learning models may include a neural network model (e.g., deep learning model), a generative adversarial network (GAN), a deep belief network (DBN), a stacked autoencoder (SAE), a logistic regression (LR) model, a support vectors machine (SVM) model, a decision tree model, a naive Bayes model, a random forest model or a restricted Boltzmann machine (RBM), a gradient boosted decision tree (GBDT) model, a LambdaMART model, an adaptive boosting model, a hidden Marker-Koff model, a perceptron neural network model, a Hopfield network model, or the like, or any combination thereof. Exemplary deep learning models may include a deep neural network (DNN) model, a convolutional neural network (CNN) model, a recurrent neural network (RNN) model, a feature pyramid network (FPN) model, or the like. Exemplary CNN models may include a V-Net model, a U-Net model, an FB-Net model, a link-Net model, or the like, or any combination thereof. The model may be trained using historical data (e.g., historical dose values and historical temperature differences), and a trained model may be generated to determine the target output dose value.

The attenuator adjustment control unit 1770 may be connected to and control the attenuator adjustment unit 1780. Further, the attenuator adjustment unit 1780 may be connected to and control the laser power attenuator 1790 to adjust the power of the output laser. In some embodiments, the laser power attenuator 1790 may be an SMA variable high-power laser attenuator. In some embodiments, the SMA variable high-power laser attenuator is configured with a power adjustment nut. The power of the output laser may be adjusted by rotating the nut in a forward direction or in a reverse direction. The attenuator adjustment control unit 1770 may determine an adjustment direction and an adjustment value of the nut (e.g., a number or count of turns in rotating the nut) based on a current output dose value and the target output dose value. In some embodiments, the temperature feedback control unit 1750, the laser dose control unit 1760, and/or the attenuator adjustment control unit 1770 may be integrated as a processing module, and the processing module operates independently relative to the control device 1710 and is connected to other components or devices of the medical treatment apparatus 1700. When the temperature measured by the temperature measurement element exceeds the preset temperature range, the processing module may determine the target output dose value of the LITT device based on the difference between the measured temperature and the preset temperature range, thus rendering the temperature measured by the temperature measurement element returns back to the preset temperature range. The processing module may be or include, for example, a microcontroller (MCU), a central processing unit (CPU), a programmable logic device (PLD), an application specific integrated circuit (ASIC), a single-chip microcomputer (SCM), a system on chip (SoC), etc. In some embodiments, the temperature feedback control unit 1750, the laser dose control unit 1760, and/or the attenuator adjustment control unit 1770 may be integrated into or be part of the control device 1710.

The attenuator adjusting unit 1780 may be connect to and control the laser power attenuator 1790. The attenuator adjusting unit 1780 may control the laser power attenuator 1790 to attenuate or gain the power of input laser at a certain proportion based on instructions (e.g., the adjustment direction and adjustment value of the nut) generated by the attenuator adjusting control unit 1770. Referring to the embodiment set forth above, the laser power attenuator 1790 is the SMA variable high-power laser attenuator, and the attenuator adjustment unit 1780 may be, for example, an electric helical adjustment stepping element. The electric helical adjustment stepping element may including a micro driving motor. The nut may be adjusted according to the determined adjustment direction and adjustment value using the micro driving motor.

The laser power attenuator 1790 may adjust the current output dose value to the target output dose value by performing an attenuation or gain on the power of the input laser at a certain proportion. The laser power attenuator 1790 may perform an attenuation at a proportion of, e.g., 10%, 20%, 30%, 40%, 50%, 60%, 70%, 80%, or 90%, or a gain at a proportion of, e.g., 10%, 20%, 30%, 40%, 50%, 60%, 70%, 80%, 90%, 100%, 120%, 150%, etc., on the power of the input laser (i.e., an attenuation/gain processing on the power of the input laser). The laser power attenuator 1790 may adjust the current output dose value dynamically, so that the temperature measured by the temperature measurement element is always within the preset temperature range.

Via the temperature feedback control unit 1750, the laser dose control unit 1760, the attenuator adjustment control unit 1770, and the attenuator adjustment unit 1780, the laser power attenuator 1790 may adjusts the current output dose value dynamically, so that the temperature measured by the temperature element is always in the preset temperature range, and the power of the laser output by the laser device of the LITT device is equal to or close to the target output dose value. The laser output by the laser device of the LITT device is transmitted to the LITT photon ablation probe 1742 through the optical relaying and processing device 1724, thereby achieving the purpose of adjusting and adapting ablation energy of the laser. The operations among the above modules, units or elements may form a dynamic adjustment process. By performing the operations in one or more reciprocating cycles, the temperature measured by the LITT photon thermometric probe 1744 (e.g., the temperature of a farthest position at the edge of the target object) is dynamically maintained within the preset temperature range. An electrical connection is established between any two units among the units mentioned above, and a temperature control signal is transmitted stably and in real time in the form of an electric signal. In combination with the high accuracy of the LITT photon thermometric probe 1744 (which is more accurate than the MRTI), thereby improving an accuracy of the temperature control and timeliness of the feedback adjustment, and facilitating the temperature maintenance at the farthest edge of the target object relative to the LITT photon ablation probe 1742 within the preset temperature range, thus ensuring the therapeutic effect.

FIG. 18 is a schematic diagram of an LITT photon thermometric probe according to some embodiments of the present disclosure.

The LITT photon thermometric probe 1805 is disposed in a probe sleeve. The probe sleeve is made of a specific material, such as polyetheretherketone (PEEK), that has high temperature resistance and corrosion resistance. The LITT photon thermometric probe 1805 may be a FBG thermometric probe, also referred to as FBG sensor. The FBG thermometric probe includes an optical fiber made of specific raw material. The specific raw material may be exposed to ultraviolet light in a certain wavelength range (e.g., 240-244 nm, 244-248 nm, 248-252 nm, 252-256 nm, etc.). Exemplarily, the specific raw material for preparing the FBG thermometric probe may be fixed on a fixing device (e.g., a clamp), and the fixing device is fixedly connected to a motion driver. A laser device, controlled by a laser device controller, emits laser. The emitted laser passes through at least one beam correction device (e.g., two oppositely arranged excimer laser 45° reflecting mirrors with a characteristic wavelength of 248 nm), a slit diaphragm (e.g., a slit diaphragm having a width of 4.5 mm), a ultraviolet coated lens (e.g., an ultraviolet coated fused quartz plano-convex cylindrical lens with a characteristic wavelength of 245-440 nm), and a phase mask (e.g., 1460-1600 nm ultra-bandwidth phase mask of ultraviolet radiation with a 248 nm characteristic wavelength) sequentially, and form a striped light spot on a surface of the specific raw material. Then, the motion driver, controlled by the motion drive controller, drives the raw material to move. During the motion of the raw material, the raw material is irradiated by the laser, and forms the FBG thermometric probe.

During a treatment, a driving motor 1810 (e.g., the second driving device as set forth above) may provide a driving force. The driving force may be transferred to a single-axis three-dimensional framework 1820 (e.g., the single-axis three-dimensional framework 1738) via a driving cable 1815 (e.g., the second translation cable) that supports a translational motion such as moving forward or retreating. In such a case, the LITT photon thermometric probe 1805 may be driven to move forward or retreat, and arrive at a position at the edge of the target object (e.g., the position as shown in FIG. 20). In this process, a motion sensor (not shown in the figure), disposed on the single-axis three-dimensional framework 1820, may monitor the position of the LITT photon thermometric probe 1805. Sensing signals of the motion sensor is transmitted through a motion sensor cable 1825. An optical signal measured by the LITT photon thermometric probe 1805, in the form of a temperature sensing light spectrum, is sent to the temperature feedback control unit 1750 in real time through a LITT photon thermometric probe channel 1830. An integrating element 1835 provides an interface support for the LITT photon thermometric probe channel 1830 to be mounted on a portable connector.

FIG. 19 is a schematic diagram of an LITT photon ablation probe according to some embodiments of the present disclosure.

As illustrated in FIG. 19, the LITT photon ablation probe may be an LITT photon lateral ablation probe 1910 or an LITT photon circumferential ablation probe 1920. In some embodiments, the LITT photon lateral ablation probe 1910 may include an OCT probe 1912 and an LITT lateral ablation probe 1914. The LITT photon circumferential ablation probe 1920 may include an OCT probe 1922, and an LITT circumferential ablation probe 1924. Each of the LITT photon lateral ablation probe 1910 and the LITT photon circumferential ablation probe 1920 may include a probe sleeve made of a specific material, such as polyetheretherketone (PEEK) that has high temperature resistance and corrosion resistance.

The LITT lateral ablation probe 1914 includes a probe main body, a connection surface, and a coating layer. The connection surface is a joint interface between the probe main body and the coating layer. The probe main body has a shape of a cylinder. The probe main body includes a probe core and a hard cladding structure located at an outer peripheral of the probe core. The probe core is made of pure silicon oxide material. The hard cladding structure is made of TECS material. In some embodiments, an end (far end) of the probe main body has an angle of inclination (i.e., an end surface of the probe main body and an axis of the probe main body form a certain angle, and the angle is an acute angle). The connection surface is formed by polishing the end surface of the probe main body. The coating layer uses noble metal (e.g., gold, silver, and metal of the platinum family) target material. In some embodiments, the coating layer may be formed by performing magnetron sputtering coating on the connection surface.

In some embodiments, the coating layer of the LITT lateral ablation probe 1914 may be replaced with a lens (e.g., a sapphire lens). In such a case, an end (far end) of the probe main body may be a flat surface or have an angle of inclination (i.e., an end surface of the probe main body is perpendicular to the axis of the probe main body or form a certain angle with the axis of the probe main body, and the angle is an acute angle). The connection surface and the lens are connected through fusion welding using electric arc welding or melted tungsten (such as tungsten wires), iridium (such as iridium wires), etc., at a high temperature. As for the fusion welding using the melted tungsten or iridium at the high temperature, a fire polishing processing may be performed on the tungsten or iridium, i.e., a welded surface between the probe main body and the lens is processed using the fire polishing.

The LITT circumferential ablation probe 1924 may include a probe main body. The probe main body includes a probe core and a hard cladding structure located at an outer peripheral of the probe core. The probe core is made of pure silicon oxide material. The hard cladding structure is made of TECS material. A far end (an end which is closer to the target object) of the probe main body includes a cone-shaped surface. A diameter of the cone-shaped surface reduces from an initial diameter to a preset diameter gradually. One or more grooves are set on the cone-shaped surface, which are carved using an optoelectronic device. Laser transmitted to the LITT circumferential ablation probe may be emitted out of the one or more grooves. The one or more grooves are evenly (dispersively) distributed on the cone-shaped surface in an arabesquitic pattern (e.g., a thread pattern), and do not overlap. The arabesquitic pattern of the one or more grooves includes a single thread pattern (bostrychoid), a cross-thread pattern (e.g., dual-thread crossing pattern), a rhombic grid pattern, a honeycomb pattern, etc., or a combination thereof. In this embodiment, the LITT lateral ablation probe 1914 and LITT circumferential ablation probe 1924 may be the same as or similar to the LITT lateral ablation probes 700, 800 and the LITT circumferential ablation probe 900, respectively, which are described in FIGS. 6-11, and are not repeated here.

During a treatment, a driving motor 1930 (e.g., the first driving device as set forth above) may provide a driving force. The driving device may be transferred to a dual-axis three-dimensional framework 1945 (e.g., the dual-axis three-dimensional framework 1736) via a driving cable 1935 (e.g., the first translation cable) that supports a translational motion such as moving forward or retreating and a driving cable 1940 (e.g., the first rotation cable) that supports a rotational motion. In such a case, the LITT photon ablation probe (e.g., the LITT photon lateral ablation probe 1910 or the LITT photon circumferential ablation probe 1920) may be driven to translate and rotate with two degrees of freedom, arrive at a position where the target object is located (e.g., the position as shown in FIG. 20), and perform an LITT radiation on the target object. In this process, a motion sensor (not shown in the figure), disposed on the dual-axis three-dimensional framework 1945, may monitor the position of the LITT photon ablation probe. Sensing signals of the motion sensor is transmitted through a motion sensor cable 1950.

When the LITT photon lateral ablation probe 1910 is used, the LITT lateral ablation probe 1914 may receive ablation laser at a specific power (e.g., 1-50 W, 1-20 W, 1-10 W, 1-8 W) transmitted through an LITT ablation probe channel 1955. By placing the LITT photon lateral ablation probe 1910 at a lateral side of the target object, the ablation laser may emit towards the target object in a certain direction. In the meanwhile, the OCT probe 1912 in the LITT photon lateral ablation probe 1910 may be used for scanning and imaging of the ablation condition or pathological diagnosis of the target object in real time, and transmit the scanning and imaging result(s) to the OCT control module 1711 through an OCT probe channel 1960 in real time. The OCT probe channel 1960 and the LITT ablation probe channel 1955 are integrated (e.g., mechanically coupled) via an integrating element 1965.

When the LITT photon circumferential ablation probe 1920 is used, the LITT circumferential ablation probe 1924 may receive ablation laser at a specific power (e.g., 1-50 W, 1-20 W, 1-10 W, 1-8 W). The ablation laser may emit towards the target object (e.g., cancerous tissue) dispersively through the one or more grooves set on the LITT circumferential ablation probe 1924. In the meanwhile, the OCT probe 1922 in the LITT photon circumferential ablation probe 1920 may be used for scanning and imaging of the ablation condition or pathological diagnosis of the target object in real time, and transmit the scanning and imaging result(s) to the OCT control module 1711 through the OCT probe channel 1960 in real time.

FIG. 20 is a schematic diagram of an LITT photon ablation probe and an LITT photon thermometric probe during a treatment according to some embodiments of the present disclosure.

In this embodiment, the LITT photon thermometric probe 2010 and the LITT photon ablation probe (LITT photon circumferential ablation probe 2020 or LITT photon lateral ablation probe 2030) may be placed at preset positions before LITT photon therapy is initiated. The LITT photon thermometric probe 2010 may acquire a temperature of a specific position at the edge of the target object. The specific position may be a position that is located on the edge of the target object and farthest from the LITT photon ablation probe. A distance between the LITT photon thermometric probe 2010 and the LITT photon ablation probe is smaller than the size of the target object. A region within the edge of the target object may have, for example, an irregular shape. The target object can be deemed as an equivalent circle. For example, a minimum circumscribed circle of the target object may be determined. The minimum circumscribed circle may be designated as the equivalent circle of the target object. The size of the target object may be, for example, a diameter of the equivalent circle.

Merely for illustration purposes, as shown in the figure, if the LITT photon ablation probe is the LITT photon circumferential ablation probe 2020, the LITT photon circumferential ablation probe 2020 (including the LITT circumferential ablation probe) may be set at an equivalent center of the target object (e.g., a center of the equivalent circle), and the LITT photon thermometric probe 2010 may be set at a position on the edge of the target object and farthest from the LITT photon circumferential ablation probe 2020. A distance between the LITT photon circumferential ablation probe 2020 and the LITT photon thermometric probe 2010 may be L_{dispersion}. The L_{dispersion} is equal to or close to the equivalent radius of the target object. The equivalent radius herein may be the radius of the minimum circumscribed circle of the edge of the target object. In combination with the (circumferential) dispersion uniformity of the ablation laser emitted out of the LITT photon circumferential ablation probe 2020, by setting the LITT photon circumferential ablation probe 2020 at the equivalent center, and setting the LITT photon thermometric probe 2010 at a position on the edge of the target object and farthest from the LITT photon circumferential ablation probe 2020, the temperature of the entire target object may be higher than the preset temperature range, thereby ensuring the accuracy of temperature measurement, and the temperature decreases from the equivalent center to the edge of the target object gradually, thus ensuring the therapeutic effect of the medical treatment apparatus 1700.

If the LITT photon ablation probe is the LITT photon lateral ablation probe 2030, the LITT photon lateral ablation probe 2030 (including the LITT lateral ablation probe) may be set at a lateral side on the edge of the target object, and the LITT photon thermometric probe 2010 may be set at an opposite side of the LITT photon lateral ablation probe 2030 on the edge of the target object and farthest from the LITT photon lateral ablation probe 2030. A distance between the LITT photon lateral ablation probe 2030 and the LITT photon thermometric probe 2010 may be Lₗₐₜₑᵣₐₗ. Lₗₐₜₑᵣₐₗ is equal to or close to the equivalent diameter of the target object. The equivalent diameter herein may be the diameter of the minimum circumscribed circle of the edge of the target object. In combination with the directivity of the ablation laser emitted out of the LITT photon lateral ablation probe 2030 in a specific direction and the uniformity within a specific direction range, by setting the LITT photon lateral ablation probe 2030 at a lateral side on the edge of the target object, and setting the LITT photon lateral ablation probe 2030 at an opposite side of the LITT photon lateral ablation probe 2030 on the edge of the target object and farthest from the LITT photon lateral ablation probe 2030, the temperature of the entire target object may be higher than the preset temperature range, thereby ensuring the accuracy of temperature measurement, and the temperature decreases from the equivalent center to the edge of the target object gradually, thus ensuring the therapeutic effect of the medical treatment apparatus 1700.

The edge of the target object may be determined by processing one or more MRI images containing the target object using an image recognition or segmentation algorithm/model. Merely for illustration, exemplary image recognition or segmentation algorithms may include a convolution algorithm, that is, a convolution operation performed on image data with a specific operator so as to determine the edge in an image. The specific operator may include a Roberts operator, a Sobel operator, a Prewitt operator, and a Laplacian operator based zero-crossing Gaussian operator. In some embodiments, the edge recognition algorithm may further include a visual feature algorithm such as a Canny detector, a boosted edge learning (BEL) algorithm, or the like.

Exemplary image recognition or segmentation models may include neural network models (e.g., deep learning models), generative adversarial networks (GAN), deep belief networks (DBN), stacked autoencoders (SAE), logistic regression (LR) models, support vector machine (SVM) models, decision tree models, naive Bayes models, random forest models or restricted Boltzmann machine (RBM), gradient boosting decision tree (GBDT) models, Lambda MART models, adaptive augmented models, hidden Markov models, perceptron neural network models, Hopfield network models, or the like, or any combination thereof. In some embodiments, the image recognition or segmentation model is trained using training samples including edges of target objects in historical images.

After the LITT photon thermometric probe 2010 and the LITT photon ablation probe are placed at the preset positions, the temperature of the tissue of the target object measured by the LITT photon thermometric probe 2010 may be within the preset temperature range (for example, 46±1° C.) by using the temperature feedback control unit 1750, the laser dose control unit 1760, the attenuator adjustment control unit 1770, the attenuator adjustment unit 1780, and the laser power attenuator 1790. Taking the LITT photon circumferential ablation probe 2020 as an example, the LITT circumferential ablation probe in the LITT photon circumferential ablation probe 2020 delivers ablation laser with a power of 1-8 W and a characteristic wavelength of 1064 nm, by controlling the LITT photon thermometric probe 2010 to detect the temperature at the preset position and controlling the temperature feedback control unit 1750, laser dose control unit 1760, attenuator adjustment control unit 1770, attenuator adjustment unit 1780 and laser power attenuator 1790 to feedback control the LITT circumferential ablation probe, the temperature of the entire target object is within the preset temperature range. In this way, an overall temperature of the target object may be set to be equal to or higher than the preset temperature range accurately, which facilitate a better therapeutic effect on metastatic (spread) cancer. Besides, during a treatment, the OCT probe in the LITT photon lateral ablation probe 1910 may be used for scanning and imaging of the ablation condition or pathological diagnosis of the target object in real time, and further facilitate a pathological evaluation, thereby improving the treatment success rate and treatment efficiency of the medical treatment apparatus 1700.

At this time, the temperature of tumor tissue adjacent to the LITT photon circumferential ablation probe 2020 may rise to nearly 100° C., and coagulate and necrosis rapidly, while cells in an annular region at an outer peripheral of the tumor tissue may apoptos slowly for 48-72 hours or longer. The laser is absorbed continuously within the tissue, which in turn generates continuous heat. The high temperature close to the LITT photon circumferential ablation probe 2020 causes rupture of cell membranes and continued coagulation of proteins, resulting in instantaneous necrosis of affected tissue continuously. The temperature closer to the temperature probe 2010 is lower and is causing a rim of apoptotic cells called the transition zone which is known to generate immunogenic cell death. Ultimately, within a duration of several hours to several days, these changes result in a complete destroy of the target cancerous tissue. The dying cancerous tissue cells in the transition region induce cell rupture to initiate apoptosis of cancer cells through the head ablation, resulting in a release of antigen factors, and causing an immune response of human body.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electromagnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

What is claimed is:

1. A laser interstitial thermal therapy (LITT) lateral ablation probe, which is used in an LITT device, wherein the LITT lateral ablation probe comprises:
a probe main body; and
a coating layer covered on an end surface of the probe main body, wherein
the end surface of the probe main body and an axis of the probe main body form a first angle, the probe main body comprises a probe core and a hard cladding structure located at an outer peripheral of the probe core, the probe core is made of pure silicon oxide material, the hard cladding structure is made of technology enhanced clad silica (TECS) material, and the coating layer comprises noble metal.

2. The LITT lateral ablation probe of claim 1, wherein the coating layer has a dual-layer structure, a layer of the dual-layer structure which is close to the end surface of the probe main body comprises pure silver, and the other layer of the dual-layer structure which is away from the end surface of the probe main body comprises silicon monoxide.

3. The LITT lateral ablation probe of claim 1, wherein the LITT lateral ablation probe is obtained by brightening and cleaning the end surface of the probe main body, and then further covering the coating layer on the end surface through magnetron sputtering.

4. A laser interstitial thermal therapy (LITT) lateral ablation probe, which is used in an LITT device, wherein the LITT lateral ablation probe comprises:
a probe main body; and
a lens connected to an end surface of the probe main body, wherein
the end surface of the probe main body is perpendicular to an axis of the probe main body, the lens has a first vertex angle, the probe main body comprises a probe core and a hard cladding structure located at an outer peripheral of the probe core, and the lens is a sapphire lens.

5. The LITT lateral ablation probe of claim 4, wherein the lens is a chamfered cylindrical lens, or a chamfered hemi-spherical or hemi-ellipsoidal lens.

6. The LITT lateral ablation probe of claim 4, wherein the LITT lateral ablation probe is obtained by brightening and cleaning the end surface of the probe main body, connecting the probe main body and the lens through fusion welding, and processing a welded surface between the probe main body and the lens using fire polishing.

7. A laser interstitial thermal therapy (LITT) circumferential ablation probe, which is used in an LITT device, wherein the LITT circumferential ablation probe comprises:
a probe main body, the main body of the probe comprising a cone-shaped surface, one or more grooves being set on the cone-shaped surface, the one or more grooves being evenly distributed on the cone-shaped surface in an arabesquitic pattern, wherein the LITT circumferential ablation probe emits laser out of the one or more grooves.

8. The LITT circumferential ablation probe of claim 7, wherein a diameter of the cone-shaped surface decreases from an initial diameter to a preset diameter, the initial diameter is 630 μm, and the preset diameter is 100 μm.

9. The LITT circumferential ablation probe of claim 7, wherein the arabesquitic pattern comprises a single thread pattern, a cross-thread pattern, or a rhombic grid pattern, or a combination thereof.

10. The LITT circumferential ablation probe of claim 7, wherein the cone-shaped surface is formed by performing operations comprising:
fixing two ends of a first workpiece with cone-shaped surface to be carved on a sliding rail and a fixing device, respectively;
controlling, by a laser device controller, a laser device, to emit laser, wherein a power of the emitted laser is 30 W, a wavelength of the emitted laser is 10600 nm, the emitted laser is attenuated to 6.6-11.2 W by a laser power attenuator, the attenuated laser is split into two laser beams by a diffraction splitting lens unit, and the two laser beams have equal power and are focused onto a surface of the first workpiece by a focusing lens unit; and
driving, by a motion driver, the first workpiece to move, the motion driver being controlled by a motion driver controller.

11. The LITT circumferential ablation probe of claim 7, wherein the one or more grooves that are evenly distributed on the cone-shaped surface in the arabesquitic pattern are formed by performing operations comprising:
fixing two ends of a second workpiece with arabesquitic pattern to be carved on a sliding rail and a fixing device, respectively;
controlling, by a laser device controller, a laser device, to emit laser, wherein a power of the emitted laser is 30 W, a wavelength of the emitted laser is 10600 nm, the emitted laser is focused on a surface of the second workpiece by a lens set, the lens set comprises a first concave lens, a first convex lens, and a second concave lens, the first concave lens and the first convex lens are used to expand a diameter of the emitted laser into a first diameter, the second concave lens is used to focus the laser beam of the first diameter to form a spot on the surface of the second workpiece, and the spot has a diameter of 45 μm; and
driving, by a motion driver, the second workpiece to move, the motion driver being controlled by a motion driver controller.

12. The LITT circumferential ablation probe of claim 7, wherein a spatial intensity distribution of the laser emitted by the LITT circumferential ablation probe is obtained through operations comprising:
obtaining signals output by a laser measurement sensor, and generating a polar intensity of a specific orientation of the laser emitted by the LITT circumferential ablation probe, wherein the LITT circumferential ablation probe is connected to a helium-neon laser device that generates laser with a wavelength of 632.8 nm, a slit diaphragm is set between the laser measurement sensor and the LITT circumferential ablation probe, and a slit size of the slit diaphragm is 0.3 mm; and
determining the spatial intensity distribution of the laser emitted by the LITT circumferential ablation probe by driving, by a motion driver, the slit diaphragm and the laser measurement sensor to move along a circumferential direction and an axial direction of the cone-shape surface of the LITT circumferential ablation probe, the motion driver being controlled by a motion driver controller, wherein the slit diaphragm and the laser measurement sensor are fixedly connected to the motion driver.

13. A medical treatment apparatus, comprising:
a magnetic resonance imaging (MRI) device, configured for imaging of a specific region comprising a target object and generating a magnetic resonance image;
a laser interstitial thermal therapy (LITT) device, comprising:
an LITT probe, the LITT probe being positioned close to the target object based on the magnetic resonance image, and configured to treat the target object by emitting laser; and
a temperature measurement element, the temperature measurement element and the LITT probe being integrated as an integrated probe, and the temperature measurement element being configured to obtain a temperature of the target object, wherein the LITT probe comprises an LITT lateral ablation probe or an LITT circumferential ablation probe.

14. The medical treatment apparatus of claim 13, wherein the LITT lateral ablation probe comprises:
a probe main body; and
a coating layer covered on an end surface of the probe main body, wherein
the end surface of the probe main body and an axis of the probe main body form a first angle, the probe main body comprises a probe core and a hard cladding structure located at an outer peripheral of the probe core, and the coating layer comprises noble metal.

15. The medical treatment apparatus of claim 14, wherein the coating layer has a dual-layer structure, a layer of the dual-layer structure which is close to the end surface of the probe main body comprises pure silver, and the other layer of the dual-layer structure which is away from the end surface of the probe main body comprises silicon monoxide.

16. The medical treatment apparatus of claim 14, wherein the LITT lateral ablation probe is obtained by brightening and cleaning the end surface of the probe main body, and then further covering the coating layer on the end surface through magnetron sputtering.

17. The medical treatment apparatus of claim 13, wherein the LITT lateral ablation probe comprises:
a probe main body; and
a lens connected to an end surface of the probe main body, wherein
the end surface of the probe main body is perpendicular to an axis of the probe main body, the lens has a first vertex angle, the probe main body comprises a probe core and a hard cladding structure located at an outer peripheral of the probe core, and the lens is a sapphire lens.

18. The medical treatment apparatus of claim 17, wherein the lens is a chamfered cylindrical lens, or a chamfered hemi-spherical or hemi-ellipsoidal lens.

19. The medical treatment apparatus of claim 17, wherein the LITT lateral ablation probe is obtained by brightening and cleaning the end surface of the probe main body, connecting the probe main body and the lens through fusion welding, and processing a welded surface between the probe main body and the lens using fire polishing.

20. The medical treatment apparatus of claim 13, wherein the LITT circumferential ablation probe comprises:
a probe main body, the main body of the probe comprising a cone-shaped surface, one or more grooves being set on the cone-shaped surface, the one or more grooves being evenly distributed on the cone-shaped surface in an arabesquitic pattern, wherein the LITT circumferential ablation probe emits laser out of the one or more grooves.

21. The medical treatment apparatus of claim 20, wherein a diameter of the cone-shaped surface decreases from an initial diameter to a preset diameter, the initial diameter is 630 μm, and the preset diameter is 100 μm.

22. The medical treatment apparatus of claim 20, wherein the arabesquitic pattern comprises a single thread pattern, a cross-thread pattern, or a rhombic grid pattern, or a combination thereof.

23. The medical treatment apparatus of claim 20, wherein the cone-shaped surface is formed by performing operations comprising:
fixing two ends of a first workpiece with cone-shaped surface to be carved on a sliding rail and a fixing device, respectively;
controlling, by a laser device controller, a laser device, to emit laser, wherein a power of the emitted laser is 30 W, a wavelength of the emitted laser is 10600 nm, the emitted laser is attenuated to 6.6-11.2 W by a laser power attenuator, the attenuated laser is split into two laser beams by a diffraction splitting lens unit, and the two laser beams have equal power and are focused onto a surface of the first workpiece by a focusing lens unit; and
driving, by a motion driver, the first workpiece to move, the motion driver being controlled by a motion driver controller.

24. The medical treatment apparatus of claim 20, wherein the one or more grooves that are evenly distributed on the cone-shaped surface in the arabesquitic pattern are formed by performing operations comprising:
fixing two ends of a second workpiece with arabesquitic pattern to be carved on a sliding rail and a fixing device, respectively;
controlling, by a laser device controller, a laser device, to emit laser, wherein a power of the emitted laser is 30 W, a wavelength of the emitted laser is 10600 nm, the emitted laser is focused on a surface of the second workpiece by a lens set, the lens set comprises a first concave lens, a first convex lens, and a second concave lens, the first concave lens and the first convex lens are used to expand a diameter of the emitted laser into a first diameter, the second concave lens is used to focus the laser beam of the first diameter to form a spot on the surface of the second workpiece, and the spot has a diameter of 45 μm; and
driving, by a motion driver, the second workpiece to move, the motion driver being controlled by a motion driver controller.

25. The medical treatment apparatus of claim 20, wherein a spatial intensity distribution of the laser emitted by the LITT circumferential ablation probe is obtained through operations comprising:
obtaining signals output by a laser measurement sensor, and generating a polar intensity of a specific orientation of the laser emitted by the LITT circumferential ablation probe, wherein the LITT circumferential ablation probe is connected to a helium-neon laser device that generates laser with a wavelength of 632.8 nm, a slit diaphragm is set between the laser measurement sensor and the LITT circumferential ablation probe, and a slit size of the slit diaphragm is 0.3 mm; and
determining the spatial intensity distribution of the laser emitted by the LITT circumferential ablation probe by driving, by a motion driver, the slit diaphragm and the laser measurement sensor to move along a circumferential direction and an axial direction of the cone-shape surface of the LITT circumferential ablation probe, the motion driver being controlled by a motion driver controller, wherein the slit diaphragm and the laser measurement sensor are fixedly connected to the motion driver.
